(19) United States
(12) Patent Application Publication
(10) Pub. No.: US 2020/0207989 A1
(43) Pub. Date: Jul. 2, 2020

(54) FLUORESCENT PROBE FOR ALDH3A1 DETECTION

(71) Applicant: The University of Tokyo, Tokyo (JP)

(72) Inventors: Yasuteru URANO, Tokyo (JP); Tasuku UENO, Tokyo (JP); Katsuya TSUCHIHARA, Chiba (JP); Atsushi YAGISHITA, Chiba (JP)

(73) Assignee: The University of Tokyo, Tokyo (JP)

(21) Appl. No.: 16/466,190

(22) PCT Filed: Dec. 6, 2017
(86) PCT No.: PCT/JP2017/043881

§ 371 Date: Jan. 22, 2020

(30) Foreign Application Priority Data
Dec. 7, 2016 (JP) 2016-237366

(51) Int. Cl. C09B 11/28; C09B 23/00; C09B 57/00; G01N 21/64; G01N 33/58

(52) U.S. Cl. CPC C09B 11/28; C09B 23/00; C09B 57/00; G01N 15/14; G01N 33/582; G01N 2333/90203; G01N 21/6428

(57) ABSTRACT

[Problem to be Solved] To provide a fluorescent probe for ALDH3A1 detection that can be used in flow cytometry adaptable to live cells.

[Means of Resolution] A compound represented by general formula (I) or a salt thereof, wherein the compound or salt thereof has a retention time on an HPLC chromatogram measured under the following conditions of longer than 6.9 minutes when said compound is in aldehyde form and of 6.9 minutes or less when said compound is in carboxylic acid form.

### TECHNICAL FIELD

The present invention relates to a novel fluorescent probe, more specifically to a fluorescent probe capable of detecting ALDH3A1.

### BACKGROUND ART

ALDH is an enzyme that oxidizes various aldehydes to carboxylic acids. Human ALDH is known to have 19 isoforms, each having a substrate belonging thereto (substrate specificity).

Table 1 shows the ALDH isoforms being studied relatively actively in the fields of stem cell and cancer research, and the characteristics of the ALDH isoforms.

The isoforms for which knowledge as markers is accumulating are ALDH1A1 and ALDH1A3, and there are many reports that these are stem cell markers in normal and cancer cells. Meanwhile, ALDH3A1 is being studied from the viewpoint of anticancer drug resistance because ALDH3A1 metabolizes and inactivates some anticancer drugs. There are also reports that ALDH3A1 is involved in cell proliferation based on knockdown experiments in lung cancer cell lines (Non-Patent Document 1) and reports that ALDH3A1 is involved in spheroid formation ability and further malignant transformation of cancer tissue in prostate cancer cells (Non-Patent Document 2).

TABLE 1
Main ALDH isoforms and their characteristics

| Isoform | Main substrate | Function and biology |
|---|---|---|
| ALDH1A1 | retinaldehyde | stem cell marker |
| | | deactivation of cyclophos- |
| | | phamide (anticancer drug) |
| | | metabolism of carcinogenic |
| | | aldehyde (acrolein) |
| ALDH1A3 | retinaldehyde | stem cell marker |
| ALDH2 | acetaldehyde | metabolism of carcinogenic |
| | | aldehyde (acetaldehyde) |
| | | (decreased activity involved |
| | | in esophageal cancer and |
| | | carcinogenesis in Fanconi |
| | | anemia patients} |
| | | stem cell marker |
| ALDH3A1 | aromatic and | deactivation of cyclophos- |
| | medium-chain | phamide (anticancer drug) |
| | aliphatic | metabolism of carcinogenic |
| | aldehydes | aldehyde (acrolein) |
| | | antioxidant-related actions |

Thus, ALDH3A1 is believed to have important functions, but research on ALDH3A1 has lagged greatly in comparison to ALDH1. One reason is whether or not a fluorescent probe for the ALDH exists. A fluorescent probe (ALDEFLUOR (registered trademark)) that makes it possible to distinguish ALDH1 activity in living cells, sort cells based on whether the activity is high or low, and carry out successive experiments already exists for ALDH1. Stem cell abilities include self-replication ability and differentiation ability, but experiments that demonstrate these abilities are necessary to show that ALDH1 is a stem cell marker. In this case, cells must be sorted out in accordance with high or low ALDH1 activity using live cells. In addition, when the effects of an anticancer drug are studied in vitro, there is a method for assaying live cells using a dye called MTT assay which is widely used because it is simple and also applicable to high throughput.

As described above, the magnitude of ALDH activity differs in each cell even within the same cell line, but there are many reports of a relationship between the magnitude of this ALDH activity and anticancer drug resistance (Non-Patent Document 3). It is possible to investigate the relationship between anticancer drug resistance and ALDH activity in detail by examining the distribution of ALDH activity of live cells before and after anticancer drug administration.

Thus, if there were an ALDH3A1 probe that could be adapted to live cells, research could be expanded into a variety of studies, but a useful ALDH3A1 probe has not yet been realized.

### PRIOR ART DOCUMENTS

### Non-Patent Documents

Non-Patent Document 1: Mol. Cancer 2008, 7, 87.

Non-Patent Document 2: Br. J. Cancer 2014, 110, 2593.

Non-Patent Document 3: Biomed. Pharmacother. 2013 September; 67(7): 669-80. “The role of aldehyde dehydrogenase (ALDH) in cancer drug resistance.”

### SUMMARY OF THE INVENTION

### Problems to be Solved by the Invention

The purpose of the present invention is to provide a fluorescent probe for ALDH3A1 detection that can be used in flow cytometry adaptable to live cells.

### Means Used to Solve the Above-Mentioned Problems

As a result of in-depth studies conducted to solve the above problem, the present inventors discovered that benzaldehyde is effective as a reaction substrate of ALDH3A1 and also that a fluorescent probe that reacts specifically with ALDH3A1 can be provided by linking benzaldehyde and a fluorophore by a linker having a specific structure.

Also, as the mechanism when detecting cells having high ALDH3A1 activity using the fluorescent probe, the present inventors used the principle that an aldehyde-form fluorescent probe is hydrophobic and can permeate the cell membrane but becomes water-soluble and unable to permeate the membrane when the probe is metabolized into carboxylic acid form by ALDH3A1 activity due to the negative charge of the carboxylic acid, causing the carboxylic acid-form probe which has become membrane impermeable to reside and accumulate only in cells with high ALDH3A1 activity (see FIG. 1).

Upon having undertaken a variety of investigations, the present inventors discovered that the probe as a whole has excessive membrane permeability due to the low water solubility even when metabolized into carboxylic acid form and that the carboxylic acid-form probe cannot reside and accumulate within the cells when combined with a highly hydrophobic fluorophore because the benzaldehyde of the reaction site is highly hydrophobic. Therefore, the present inventors accomplished the invention upon discovering that a fluorescent probe for ALDH3A1 detection capable of being used in flow cytometry adaptable to live cells can be provided by setting the hydrophilicity level of the fluorescent probe when in carboxylic acid form within a specific range.

Specifically, the present invention provides:

[1] A compound represented by general formula (I) or a salt thereof;

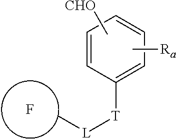

(where:
Rₐ is a hydrogen atom or a C1-4 alkyl group;
T is selected from the following divalent groups:

### —N(R_{b})—,

—C(R_{c})₂—

### —O—

(R_{b} is a C1-4 alkyl group, R_{c} is a hydrogen atom or a C1-4 alkyl group, and each R_{c} may be the same or different);
L is a linker;

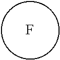

represents a fluorophore,
the fluorophore is selected from xanthene dyes, BODIPY fluorophores, or cyanine fluorophores)
wherein said compound or salt thereof has a retention time on an HPLC chromatogram measured under the following conditions of longer than 6.9 minutes when said compound is in aldehyde form and of 6.9 minutes or less when said compound is in carboxylic acid form.

(HPLC conditions: taking solvent A to be 0.01 M ammonium formate/water and solvent B to be 80% acetonitrile 0.01 M ammonium formate/water, chromatography is carried out under conditions of 20% solvent B for 2.5 minutes followed by a 5-minute linear gradient of solvent B from 20% to 100% (flow rate 500 μL/min).)

[2] The compound or salt thereof according to [1], wherein the linker is represented by Y—(S), Y is a linking group, and S, when present, is a crosslinking group.

[3] The compound or salt thereof according to [2], wherein the linking group is selected from —CONH—, —R—CONH—, —COO—, —R—COO—, —RO—, or —R—CO— (where R is a hydrocarbon group).

[4] The compound or salt thereof according to [2] or [3], wherein the crosslinking group is selected from C1-6 substituted or unsubstituted alkylene groups, —(CH₂—CH₂—O)ₘ— (m is 1 or 2), or a combination thereof.

[5] The compound or salt thereof according to any of [1]-[4], wherein

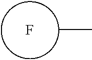

in formula (I) is represented by formula (II).

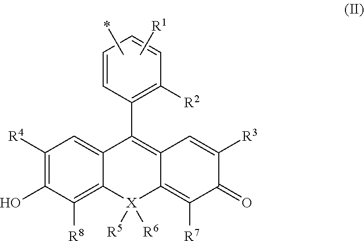

R¹ is a hydrogen atom or 1 to 4 of the same or different monovalent substituents present on the benzene ring;
R² is a hydrogen atom, a monovalent substituent, or a bond;
R³ and R⁴ are, each independently, a hydrogen atom, a C1-6 alkyl group, a halogen atom, or a bond;
R⁵ and R⁶ are, each independently, a C1-6 alkyl group, an aryl group, or a bond; however, R⁵ and R⁶ are not present when X is an oxygen atom;
R⁷ and R⁸ are, each independently, a hydrogen atom, a C1-6 alkyl group, a halogen atom, or a bond;
X is an oxygen atom or silicon atom;
* represents a site of bonding with L of formula (I) at any position on the benzene ring,
where L bonds at at least one position selected from any position on the benzene ring, any of positions R²-R⁸.)

[6] The compound or salt thereof according to [5] represented by formula (IIa).

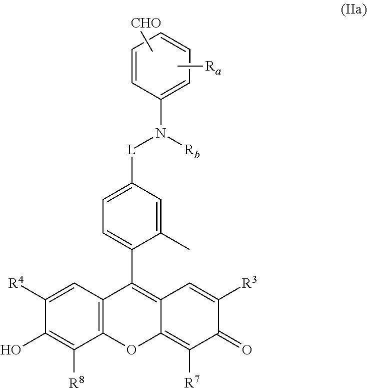

(In the formula, Rₐ, R_{b}, and L are as defined in formula (I), and R³, R⁴, R⁷, R⁸ are as defined in formula (II).)

[7] The compound or salt thereof according to any of [1]-[4], wherein

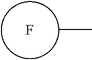

in formula (I) is represented by formula (III).

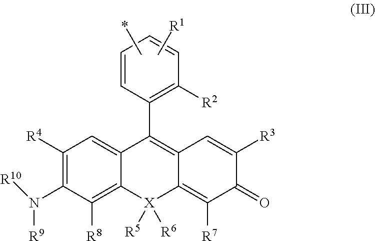

(in the formula, R¹-R⁸ and X are as defined in formula (II);

R⁹ and R¹⁰ are, each independently, a hydrogen atom, a C1-3 alkyl group, or a bond,
R⁹ and R¹⁰ together may form a 4- to 7-membered heterocyclyl including nitrogen atoms to which R⁹ and R¹⁰ are bonded,
R⁹ or R¹⁰ or both R⁹ and R¹⁰, together with R⁴ or R⁸,
respectively, may form a 5- to 7-membered heterocyclyl or heteroaryl including a nitrogen atom to which R⁹ or R¹⁰ is bonded, may also contain 1-3 heteroatoms selected from the group consisting of an oxygen atom, nitrogen atom, or sulfur atom as ring members, and the heterocyclyl or heteroaryl may also be substituted by a C1-6 alkyl, C2-6 alkenyl, or C2-6 alkynyl, C6-10 aralkyl group, or C6-10 alkyl-substituted alkenyl group;
* represents a site of bonding with L of formula (I) at any position on the benzene ring,
where L bonds at at least one position selected from any position on the benzene ring, any of positions R²-R¹⁰.)

[8] The compound or salt thereof according to any of [1]-[4], wherein

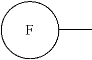

in formula (I) is represented by formula (IV).

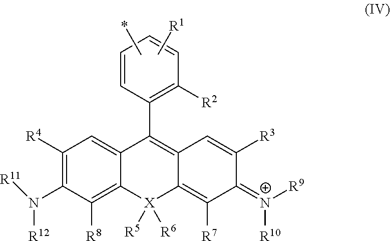

(in the formula, R¹-R⁸ and X are as defined in formula (II);

R⁹ and R¹⁰ are, each independently, a hydrogen atom or a C1-6 alkyl group,
R⁹ and R¹⁰ together may form a 4- to 7-membered heterocyclyl including nitrogen atoms to which R⁹ and R¹⁰ are bonded,
R⁹ or R¹⁰ or both R⁹ and R¹⁰, together with R³ or R⁷, respectively, may form a 5- to 7-membered heterocyclyl or heteroaryl including a nitrogen atom to which R⁹ or R¹° is bonded, may also contain 1-3 heteroatoms selected from the group consisting of an oxygen atom, nitrogen atom, and sulfur atom as ring members, and the heterocyclyl or heteroaryl may also be substituted by a C1-6 alkyl, C2-6 alkenyl, or C2-6 alkynyl, C6-10 aralkyl group, or C6-10 alkyl-substituted alkenyl group;
R¹¹ and R¹² are, each independently, a hydrogen atom, a C1-3 alkyl group, or a bond,
R¹¹ and R¹² together may form a 4- to 7-membered heterocyclyl including nitrogen atom to which R¹¹ and R¹² are bonded,
R¹¹ or R¹² or both R¹¹ and R¹², together with R⁴ or R⁸, respectively, may form a 5- to 7-membered heterocyclyl or heteroaryl including a nitrogen atom to which R¹¹ or R¹² is bonded, may also contain 1-3 heteroatoms selected from the group consisting of an oxygen atom, nitrogen atom, and sulfur atom as ring members, and the heterocyclyl or heteroaryl may also be substituted by a C1-6 alkyl, C2-6 alkenyl, or C2-6 alkynyl, C6-10 aralkyl group, or C6-10 alkyl-substituted alkenyl group;
* represents a site of bonding with L of formula (I) at any position on the benzene ring,
where L bonds at any position on the benzene ring, at at least one position selected from any of positions R2-R¹².)

[9] The compound or salt thereof according to any of [1]-[4], wherein

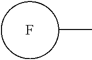

in formula (I) is represented by formula (V).

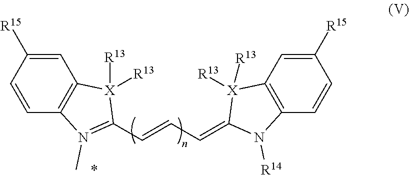

(in the formula,

R¹³ and R¹⁴ are, each independently, a hydrogen atom or a C1-6 alkyl group;
R¹⁵ is, each independently, a hydrogen atom, carboxyl group, or sulfonic acid group;
n is an integer of 1-3;
* represents a site of bonding with L of formula (I).)

[10] The compound or salt thereof according to any of [1]-[4], wherein

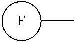

in formula (I) is represented by formula (VI).

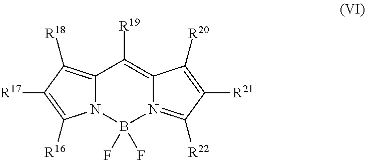

(in the formula, R¹⁶-R²² are, each independently, a hydrogen atom, a C1-6 alkyl group, a carbonyl group, an allyl group, an aryl group, a pyrrole group, a thiophene group, a furan group, a sulfonic acid group, a sulfonylamide group, a carboxyl group, a methoxy group, or a bond;

L bonds at at least one position selected from any of positions R¹⁶-R²².)

[11] A fluorescent probe for ALDH3A1 detection including the compound or salt thereof according to any of [1]-[10].

### ADVANTAGES OF THE INVENTION

The present invention makes it possible to provide a fluorescent probe for ALDH3A1 detection that can be used in flow cytometry adaptable to live cells.

The fluorescent probe of the present invention makes it possible to detect the activity of ALDH3A1 using living cells as cells. Specifically, the ALDH3A1 expression level and activity can be evaluated even by conventional techniques using ground cells, but examining the activity of ALDH3A1 with cells in a living state makes it possible to approach the essence of biology such as what will happen to high-activity cells thereafter.

### BRIEF DESCRIPTION OF THE DRAWINGS

FIG. 1A Principle of detecting cells having high ALDH3A1 activity using probe

FIG. 2 Retention times of aldehyde form and carboxylic acid form in each probe

FIG. 3 Results of cell imaging using probe 5 (scale bar 100 μm)

FIG. 4 Flow cytometry using probe 5

FIG. 5a shows the results of Western blotting six days after knockdown of OE21 cells by siRNA.

FIG. 5b shows the results of flow cytometry using knocked-down cells. (upper: assay by cells treated by negative control siRNA. lower: assay by knocked-down cells.)

FIG. 6 shows the results of Western blotting of probe 5 high-brightness cells (top) and low-brightness cells (bottom) sorted out by cell sorter using probe 5.

FIG. 7a Results of time lapse observation in the same visual field at 37° C. 40 μM of probe 5, no MK-571 (scale bar 100 μM)

FIG. 7b Results of time lapse observation in the same visual field at 37° C. 40 μM of probe 5, 200 μM of MK-571 (scale bar 100 μM)

FIG. 7c Flow cytometry. (OE21 cells, 50 μM of probe 5, 200 μM of MK-571)

FIG. 8 shows the results of flow cytometry conducted after knockdown of ALDH3A1 using siRNA against O E21 cells. (50 μM of probe 5 was used together with 200 μM of MK-571. OE21 cells with ALDH3A1 inhibited by 20 μRM of CB7 were used in the negative control.)

FIG. 9a shows Western blotting of OE21 cells with non-target shRNA (ctrl) or shRNA (KD) against ALDH3A1 introduced.

FIG. 9b shows flow cytometry using OE21 cells with shRNA introduced (50 μM of probe 5, 200 μM of MK-571).

FIG. 9c shows the results of Western blotting of a ctrl/KD=1:1 mixed sample sorted top/bottom 25% of brightness respectively by a cell sorter.

### BEST MODE FOR CARRYING OUT THE INVENTION

In the present specification, an “alkyl group” or alkyl moiety of a substituent including an alkyl moiety (such as an alkoxy group), when not mentioned in particular, means a C1-6, preferably C1-4, more preferably C1-3, linear, branched, cyclic, or combination thereof alkyl group. More specific examples include a methyl group, ethyl group, n-propyl group, isopropyl group, cyclopropyl group, n-butyl group, sec-butyl group, isobutyl group, tert-butyl group, cyclopropylmethyl group, n-pentyl group, n-hexyl group, etc., as alkyl groups.

When “halogen atom” is stated in the present specification, it may be any of a fluorine atom, chlorine atom, bromine atom, or iodine atom, preferably a fluorine atom, chlorine atom, or bromine atom.

One embodiment of the present invention is a compound represented by general formula (I) or a salt thereof.

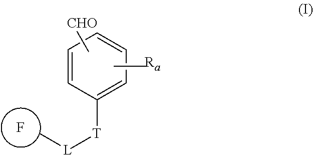

In formula (I), Rₐ represents a hydrogen atom or a C1-4 alkyl group. One to four Rₐ are present on the benzene ring, and these may be the same or different.

In one preferred embodiment of the present invention, Rₐ are all hydrogen atoms.

In formula (I), T is selected from the following divalent groups:

### —N(R_{b})—,

—C (R_{c})₂—

### —O—.

Here, R_{b} represents a C1-4 alkyl group, preferably a methyl group or ethyl group.

R_{c} represents a hydrogen atom or a C1-4 alkyl group. Each R_{c} may be the same or different.

In one preferred embodiment of the present invention, T is —N(R_{b})—.

In formula (I), L represents a linker. Examples of linkers include various substituents that link the fluorophore and a nitrogen atom.

In one preferred embodiment of the present invention, the linker is represented by X—(S); X represents a linking group and S, when present, represents a crosslinking group.

The linking group is preferably selected from an amide group (—CONH—), alkylamide group (—R—CONH—), ester group (—COO—), alkyl ester group (—R—CPP—), alkyl ether group (—RO—), or alkylcarbonyl group (—R—CO—). Here, R represents a hydrocarbon group, preferably a C1-8 alkyl.

The crosslinking group is preferably selected from a C1-6 substituted or unsubstituted alkylene group; ethylene glycol group, diethylene glycol group (i.e., —(CH₂—CH₂—O)ₘ— (m is 1 or 2)); or a combination thereof.

In formula (I), an aldehyde group (—CHO) can be introduced at any position on the benzene ring but is preferably introduced at the p position in relation to T.

In formula (I),

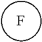

represents a fluorophore.

The fluorophore is preferably selected from xanthene dyes (Tokyo Magenta fluorophore, Tokyo Green fluorophore, rhodamine fluorophores, rhodol fluorophores), BODIPY fluorophores, or cyanine fluorophores.

In the present invention, the principle illustrated schematically in FIG. 1 was used as the mechanism when detecting cells having high ALDH3A1 activity using a fluorescent probe. In essence, the principle is that an aldehyde-form probe is hydrophobic and can permeate the cell membrane, but when metabolized to a carboxylic acid form by the activity of ALDH3A1, the probe becomes water-soluble due to the negative charge of the carboxylic acid, becoming membrane impermeable, and the carboxylic acid-form probe which has become membrane-impermeable resides and accumulates only in cells having high ALDH3A1 activity.

Here, since the benzaldehyde which is the reaction site of the compound of formula (I) of the present invention is relatively highly hydrophobic, when combined with a highly hydrophobic fluorophore, the probe as a whole has excessive membrane permeability due to the low water solubility even when metabolized to carboxylic acid form, and the carboxylic acid-form probe cannot reside and accumulate in the cells. Therefore, a fluorescent probe for ALDH3A1 detection that can be used in flow cytometry adaptable to live cells can be provided by setting the hydrophilicity level when made into carboxylic acid form within a specific range in the compound of formula (I).

In the present invention, it is preferable to use the retention time on an HPLC chromatogram as the measure of hydrophilicity.

One preferred embodiment of the present invention is a compound represented by general formula (I) or a salt thereof, wherein the compound or salt thereof has a retention time on an HPLC chromatogram measured under the following conditions of longer than 6.9 minutes when said compound is in aldehyde form and of 6.9 minutes or less when said compound is in carboxylic acid form.

HPLC conditions: taking solvent A to be 0.01 M ammonium formate/water and solvent B to be 80% acetonitrile 0.01 M ammonium formate/water, chromatography is carried out under conditions of 20% solvent B for 2.5 minutes followed by a 5-minute linear gradient of solvent B from 20% to 100% (flow rate 500 μL/min).

A C18 column (HP 3 μm, inner diameter: 2.1 mm, length: 150 mm, GL Science), for example, can be used suitably as the column.

In the present invention, any fluorophore selected from xanthene dyes (Tokyo Magenta fluorophore, Tokyo Green fluorophore, rhodamine fluorophores, rhodol fluorophores), BODIPY fluorophores, or cyanine fluorophores can be used to furnish a compound represented by formula (I) with the above HPLC chromatogram retention times.

In one aspect of the present invention,

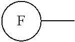

in formula (I) is represented by formula (II).

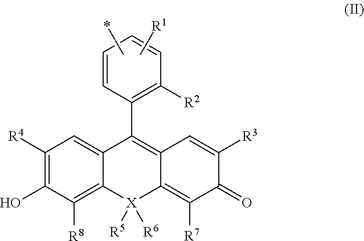

In formula (II), R¹ represents a hydrogen atom or represents from one to four of the same or different monovalent substituents present on the benzene ring.

The types of monovalent substituents represented by R¹ are not particularly limited but are preferably selected from the group consisting of C1-6 alkyl groups, C1-6 alkenyl groups, C1-6 alkynyl groups, C1-6 alkoxy groups, hydroxyl groups, carboxy groups, sulfonyl groups, alkoxycarbonyl groups, halogen atom, or amino groups. These monovalent substituents may also have one or more arbitrary substituents. For example, one or more halogen atoms, carboxy groups, sulfonyl groups, hydroxyl groups, amino groups, alkoxy groups, etc., may be present in alkyl groups represented by R¹, and alkyl groups represented by R¹ may be alkyl halide groups, hydroxyalkyl groups, carboxyalkyl groups, aminoalkyl groups, etc.

In one preferred embodiment of the present invention, R¹ are all hydrogen atoms.

In formula (II), R² represents a hydrogen atom, a monovalent substituent, or a bond. The types of monovalent substituents represented by R² are not particularly limited, but, like R¹, examples include C1-6 alkyl groups, C1-6 alkenyl groups, C1-6 alkynyl groups, C1-6 alkoxy groups, hydroxyl groups, carboxy groups, sulfonyl groups, alkoxycarbonyl groups, halogen atoms, amino groups, etc.

In one preferred embodiment of the present invention, R² is a C1-6 alkyl group, preferably a methyl group, carboxyl group, methoxy group, or hydroxymethyl group.

In formula (II), R³ and R⁴ each independently represent a hydrogen atom, C1-6 alkyl group, or halogen atom.

When R³ and R⁴ represent alkyl groups, one or more halogen atoms, carboxy groups, sulfonyl groups, hydroxyl groups, amino groups, alkoxy groups, etc., may be present in the alkyl group. For example, the alkyl group represented by R³ or R⁴ may be an alkyl halide group, hydroxyalkyl group, carboxyalkyl group, etc. R³ and R⁴ each independently are preferably a hydrogen atom or halogen atom. It is more preferred when both R³ and R⁴ are hydrogen atoms or when both R³ and R⁴ are fluorine atoms or chlorine atoms.

In formula (II), R⁵ and R⁶ each independently represent a C1-6 alkyl group, an aryl group, or a bond; however, R⁵ and R⁶ are not present when X is an oxygen atom.

When X is a silicon atom, R⁵ and R⁶ each independently are preferably a C1-3 alkyl group, and it is more preferred that both R⁵ and R⁶ are methyl groups. One or more halogen atoms, carboxy groups, sulfonyl group, hydroxyl groups, amino groups, alkoxy groups, etc., may be present in alkyl groups represented by R⁵ and R⁶, and alkyl groups represented by R⁵ and R⁶ may be alkyl halide groups, hydroxyalkyl groups, carboxyalkyl groups, etc. When R⁵ or R⁶ represents an aryl group, the aryl group may be a monocyclic aromatic group or a condensed aromatic group; and the aryl ring may include one or more ring member heteroatoms (such as a nitrogen atom, oxygen atom, or sulfur atom). A phenyl group is preferred as the aryl group. One or more substituents may be present on the aryl ring. For example, one or more halogen atoms, carboxy groups, sulfonyl groups, hydroxyl groups, amino groups, alkoxy groups, etc., may be present as substituents.

In formula (II), R⁷ and R⁸ each independently represent a hydrogen atom, a C1-6 alkyl group, a halogen atom, or a bond, the same as explained for R³ and R⁴. It is preferred that R⁷ and R⁸ are both hydrogen atoms, that both are chlorine atoms, or that both are fluorine atoms.

X represents an oxygen atom or a silicon atom. X is preferably an oxygen atom.

* represents a bonding site (bonding point, the same hereinafter) with L of formula (I) at any position on the benzene ring. Here, L can bond at any position on the benzene ring, at at least one position selected from any of positions R²-R⁸. Preferably, L can bond to any position of the benzene ring that bonds to a xanthene ring skeleton, but preferably bonds to position 4 or position 5 of the benzene ring.

One preferred embodiment of the present invention is a compound represented by formula (IIa) or a salt thereof.

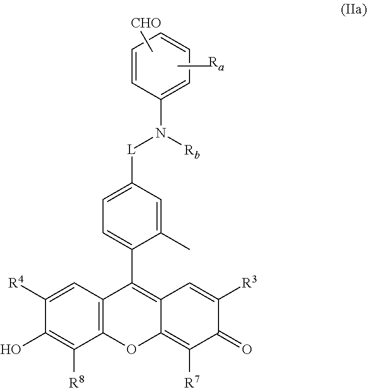

In formula (IIa), Rₐ, R_{b}, and L are as described for formula (I); R³, R⁴, R⁷, and R⁸ are as described for formula (II).

In one aspect of the present invention,

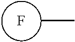

in formula (I) is represented by formula (III).

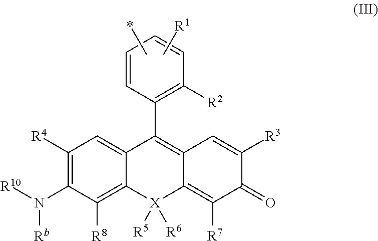

In formula (III), R¹-R⁸ and X are as defined in formula (II).

In formula (III), R⁹ and R¹⁰ each independently represent a hydrogen atom, a C1-3 alkyl group, or a bond.

Also, R⁹ and R¹⁰ together may form a 4- to 7-membered heterocyclyl including the nitrogen atoms to which R⁹ and R¹⁰ are bonded.

Also, R⁹ or R¹⁰ or both R⁹ and R¹⁰, together with R⁴ or R⁸, respectively, may form a 5- to 7-membered heterocyclyl or heteroaryl including nitrogen atoms to which R⁹ or R¹⁰ is bonded. From one to three heteroatoms selected from the group consisting of an oxygen atom, nitrogen atom, or sulfur atom may also be contained as ring members, and the heterocyclyl or heteroaryl may also be substituted by a C1-6 alkyl, C2-6 alkenyl, or C2-6 alkynyl, C6-10 aralkyl group, or C6-10 alkyl-substituted alkenyl group. Examples of the heterocyclyl or heteroaryl formed in this way include, but are not limited to, pyrrolidine, piperidine, hexamethyleneimine, pyrrole, imidazole, pyrazole, oxazole, thiazole, etc.

In formula (III), * represents a site of bonding with L of formula (I) at any position on the benzene ring. Where L bonds at at least one position selected from any position on the benzene ring, any of positions R²-R¹⁰.

Preferably, L can bond to any position of the benzene ring that bonds to a xanthene ring skeleton, but preferably bonds to position 4 or position 5 of the benzene ring.

In one aspect of the present invention,

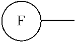

in formula (I) is represented by formula (IV).

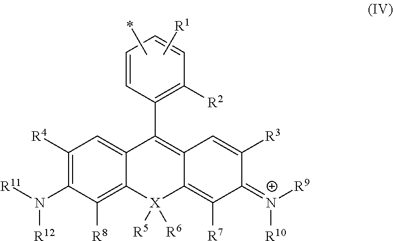

In formula (IV), R¹-R⁸ and X are as described for formula (II).

In formula (IV), R⁹ and R¹⁰ each independently represent a hydrogen atom or a C1-6 alkyl group.

Also, R⁹ and R¹⁰ together may form a 4- to 7-membered heterocyclyl including the nitrogen atoms to which R⁹ and R¹⁰ are bonded. Examples of the heterocyclyl include azetidine, pyrrolidine, etc., and these heterocyclyls may be substituted by substituents such as C1-6 alkyls, etc.

Also, R⁹ or R¹⁰ or both R⁹ and R¹⁰, together with R³ or R⁷, respectively, may form a 5- to 7-membered heterocyclyl or heteroaryl including the nitrogen atoms to which R⁹ or R¹⁰ is bonded. From one to three heteroatoms selected from the group consisting of an oxygen atom, nitrogen atom, or sulfur atom may also be contained as ring members, and the heterocyclyl or heteroaryl may also be substituted by a C1-6 alkyl, C2-6 alkenyl, or C2-6 alkynyl, C6-10 aralkyl group, or C6-10 alkyl-substituted alkenyl group. Examples of the heterocyclyl or heteroaryl formed in this way include, but are not limited to, pyrrolidine, piperidine, hexamethyleneimine, pyrrole, imidazole, pyrazole, oxazole, thiazole, etc.

In formula (IV), R¹¹ and R¹² each independently represent a hydrogen atom, a C1-3 alkyl group, or a bond.

R¹¹ and R¹² together may form a 4- to 7-membered heterocyclyl including the nitrogen atoms to which R¹¹ and R¹² are bonded. Examples of the heterocyclyl include azetidine, pyrrolidine, etc., and these heterocyclyls may be substituted by substituents such as C1-6 alkyls, etc.

Also, R¹¹ or R¹² or both R¹¹ and R¹², together with R⁴ or R⁸, respectively, may form a 5- to 7-membered heterocyclyl or heteroaryl including the nitrogen atoms to which R¹¹ or R¹² is bonded. From one to three heteroatoms selected from the group consisting of an oxygen atom, nitrogen atom, and sulfur atom may also be contained as ring members, and the heterocyclyl or heteroaryl may also be substituted by a C1-6 alkyl, C2-6 alkenyl, or C2-6 alkynyl, C6-10 aralkyl group, or C6-10 alkyl-substituted alkenyl group. Examples of the heterocyclyl or heteroaryl formed in this way include, but are not limited to, pyrrolidine, piperidine, hexamethyleneimine, pyrrole, imidazole, pyrazole, oxazole, thiazole, etc.

In formula (IV), * represents a site of bonding with L of formula (I) at any position on the benzene ring. Where L bonds at at least one position selected from any position on the benzene ring, any of positions R²-R¹².

In one aspect of the present invention,

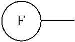

in formula (I) is represented by formula (V).

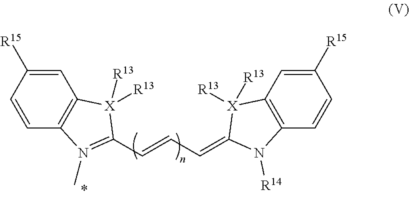

In formula (V), R¹³ and R¹⁴ each independently represent a hydrogen atom or a C1-6 alkyl group.

Also, R¹⁵ each independently represent a hydrogen atom, carboxyl group, or sulfonic acid group

In formula (V), n represents an integer of 1-3.

Also, * represents a site of bonding with L of formula (I).

In one aspect of the present invention,

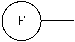

in formula (I) is represented by formula (VI).

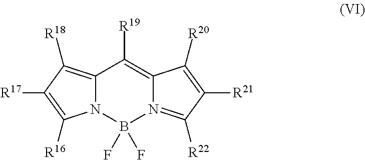

In formula (VI), R¹⁶-R²² each independently represent a hydrogen atom, a C1-6 alkyl group, a carbonyl group, an allyl group, an aryl group, a pyrrole group, a thiophene group, a furan group, a sulfonic acid group, a sulfonylamide group, a carboxyl group, a methoxy group, or a bond.

In formula (VI), L bonds at at least one position selected from any of positions R¹⁶-R²².

Compounds of the present invention sometimes have one or more asymmetrical carbons, depending on the types of substituents. Stereoisomers such as optical isomers and diastereomers can be present. Pure forms of stereoisomers, any mixtures of stereoisomers, racemates, etc., are all encompassed within the scope of the present invention. Also, compounds of the present invention represented by general formula (I) or salts thereof can also sometimes be present as hydrates or solvates. These substances are all encompassed within the scope of the present invention. The type of solvent that forms a solvate is not particularly limited; examples include solvents such as ethanol, acetone, and isopropanol.

Another embodiment of the present invention is a fluorescent probe for ALDH3A1 detection that includes a compound represented by general formula (I) that has the above HPLC chromatogram retention time, or a salt thereof.

### EXAMPLES

The present invention is explained more concretely below through examples. The scope of the present invention, however, is not limited to these examples.

### [Materials and Measurement Methods]

The reagents and solvents used in organic synthesis were supplied by Tokyo Chemical Industry, Wako Pure Chemical Industries, and Aldrich and were used without purification.

The hydrogen nuclear magnetic resonance (¹H NMR) and carbon NMR (¹³C NMR) spectra were measured by a JEOL JMN-LA300 and JMN-LA400. The mass spectra were measured by a JEOL JMS-T100LC AccuTOF.

RP-UPLC analyses were measured by a Water Acquity UPLC H-Class/Acquity UPLC PDA eλ detector/Xevo TQD quadrupole MS/MS analyzer.

LC/MS analyses were measured by an Agilent 1200 Series Diode Array Detector/6130 quadruple MS analyzer.

In imaging, images were acquired using a Leica TCS SP8. In flow cytometry, analysis was conducted using a BD FACS Canto II, and cell sorting was conducted using a BD FACS Aria.

### Synthesis Examples 1 and 2

Probe 1 and probe 2 were synthesized according to scheme 1 below.

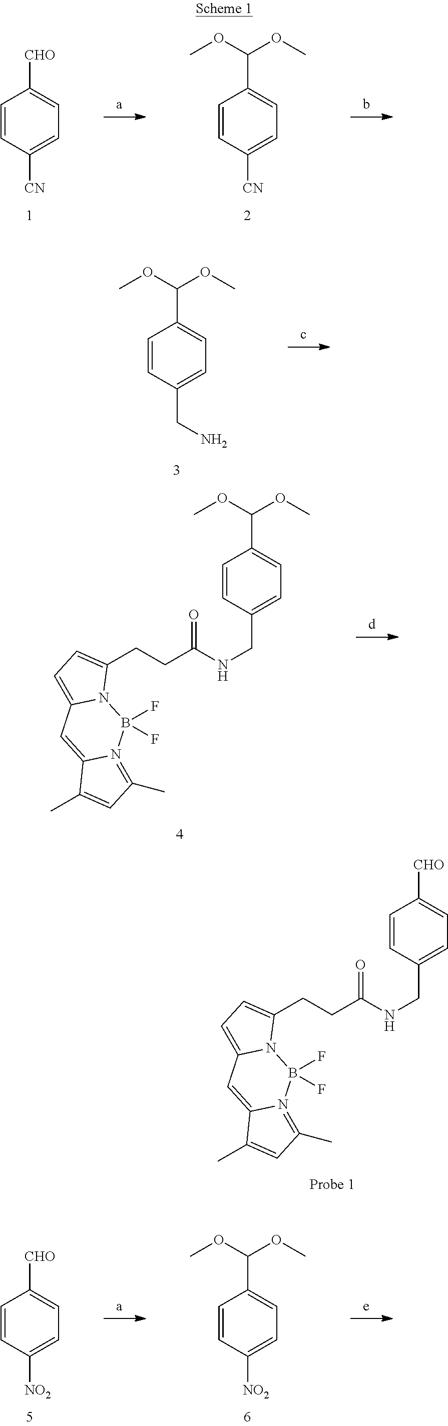

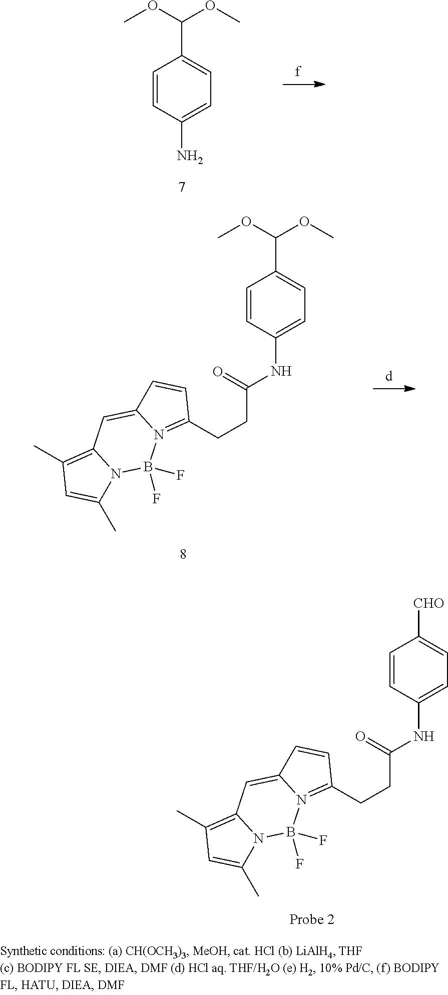

### (1) Synthesis of N-(4-dimethoxymethylbenzyl)-3-(1,3-dimethyl-4,4-difluoro-3a,4a-diaza-s-indacen-5-yl)-proionamide (Compound 4)

(2)

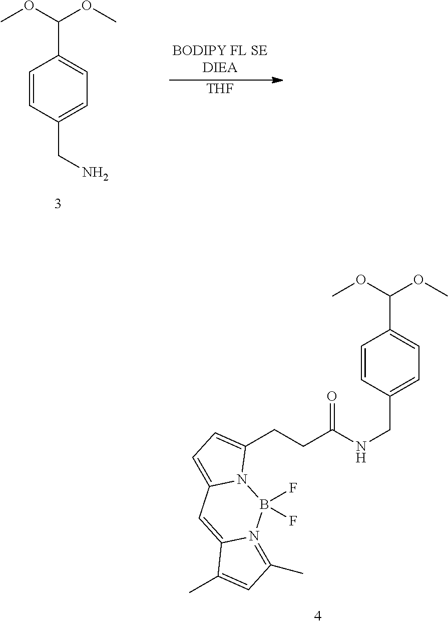

Compound 3 was synthesized from 4-cyanobenzaldehyde based on the method of the article (Giulio Cas et al. “Site-specific traceless coupling of potent cytotoxic drugs to recombinant antibodies for pharmacodelivery” J. Am. Chem. Soc., 2012, 134, pp. 5887-5892). Compound 3 (10 mg, 25.6 μmol) and 8 mL of distilled THF were added to a 50 mL round-bottomed flask. DIEA (5.4 μL, 36.5 μmol) and BODIPY FL SE dissolved in 1 mL of THF were added in the stated order to the reaction solution, heated to 40° C. on an oil bath, and stirred for three hours. After cooling to room temperature, the reaction solution was diluted using 20 mL of CH₂Cl₂, and the diluted solution was washed with 10 mL of citric acid aqueous solution (10 w/v%), 10 mL of 2 M NaHCO₃, and 10 mL of saturated saline. After the organic layer was dried using anhydrous sodium sulfate, the solution was filtered, and the filtrate was concentrated under reduced pressure. The concentrated residue was purified by column chromatography using silica gel as the carrier, and compound 4 was obtained. Admixture of acetal-deprotected compounds was seen in part of the purified product, but the purified product was used without modification in the next reaction.

### (2) Synthesis of N-(4-formylbenzyl)-3-(1,3-dimethyl-4,4-difluoro-4-bora-3a,4a-diaza-s-indacen-5-yl)-propionamide (Probe 1)

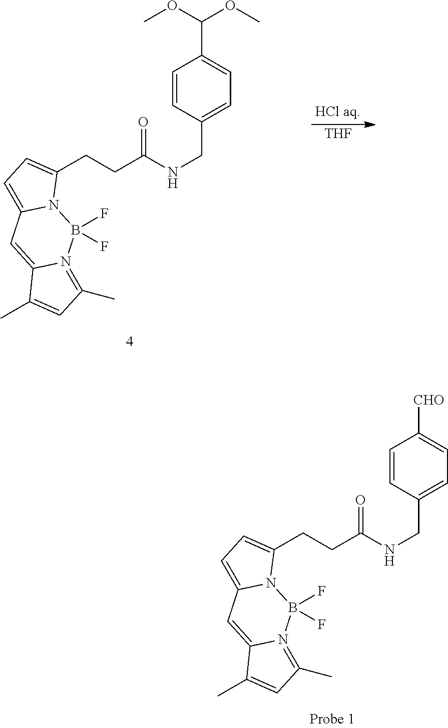

Compound 4 (crudely purified) obtained by the above method was placed in a 30 mL round-bottomed flask, and a mixed solution of 1.5 mL of distilled THF, 0.6 mL of concentrated hydrochloric acid, and 1 mL of distilled water was added thereto. After stirring vigorously for one hour at room temperature, 10 mL of saturated saline and 10 mL of AcOEt were added to the reaction solution. The solution obtained was extracted twice using 5 mL of AcOEt, and the organic layer obtained was washed with 10 mL of sodium carbonate aqueous solution and 10 mL of saturated saline. After drying the organic layer by anhydrous sodium sulfate, the solution was filtered, and the filtrate was concentrated under reduced pressure. The concentrated residue was purified by column chromatography using silica gel as the carrier, and 10 mg of probe 1 was obtained as a red solid at a yield of 98% (in two steps).

¹H-NMR (300 MHz, CD₂Cl₂): δ 2.25 (s, 3H); 2.51 (s, 3H); 2.70 (t, J=8.2 Hz, 2H); 3.25 (t, J=8.2 Hz, 2H); 4.45 (d, J=6.1 Hz, 2H); 6.15 (s, 1H); 6.25 (br, 1H); 6.28 (d, J=3.6 Hz, 1H); 6.91 (d, J=3.6 Hz, 1H); 7.14 (s, 1H); 7.32 (d, J=8.1 Hz, 2H); 7.75 (d, J=8.1 Hz, 2H); 9.94 (s, 1H). ¹³C-NMR (75 MHz, CDCl₃): δ 11.5, 15.1, 25.0, 35.9, 43.4, 117.4, 120.9, 124.3, 128.3, 128.5, 130.1, 133.7, 135.6, 135.9, 144.9.146.2, 157.4, 161.0, 171.8, 192.1. HRMS (ESI⁺): m/z calcd for C₂₂H₂₂BFN₃O₂, [M-F]⁺, 390.17891; found 390.17920 (−0.29 mmu).

### (3) Synthesis of N-(4-dimethoxymethylphenyl)-3-(1,3-dimethyl-4,4-difluoro-4-bora-3a,4a-diaza-s-indacen-5-yl)propionamide (Compound 8)

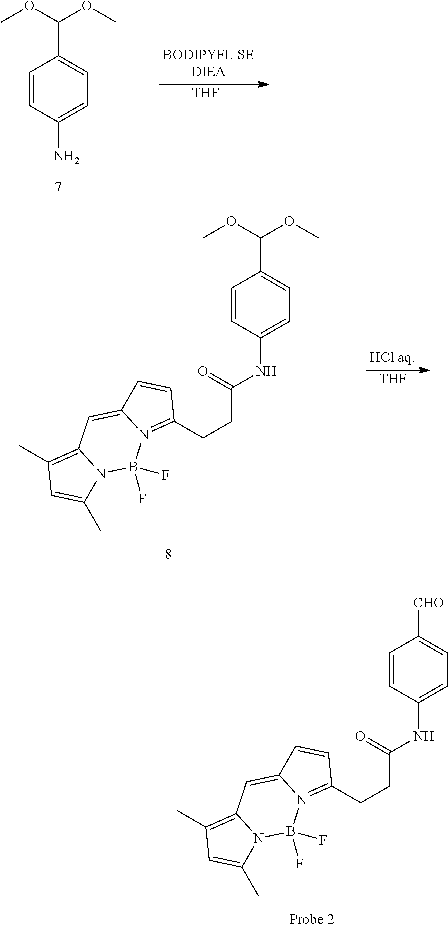

Compound 7 was synthesized according to the method of the article (Dalla Via et al. “DNA-targeting pyrroloquinoline-linked butanone and chalcones: Synthesis and biological evaluation” Eur. J. Med. Chem., 2009, 44, pp. 2854-2861). Compound 7 (2 mg, 14 μmol) and BODIPY FL (3 mg, 7 μmol) were weighed out into a 2 mL glass vial, and 150 μL of CH₂Cl₂ was added. HATU (15.6 mg, 39.5 μmol) and DIEA (50 μL) were added to the solution and stirred overnight at room temperature in an argon atmosphere. The solution obtained was purified directly by column chromatography using silica gel as the carrier without conducting post-treatment, and compound 8 was obtained as a red solid. Admixture of acetal-deprotected compounds was seen in part of the purified product, but the purified product was used without modification in the next reaction.

### (4) Synthesis of N-(4-formylphenyl)-3-(1,3-dimethyl-4,4-difluoro-4-bora-3a,4a-diaza-s-indacen-5-yl)-propionamide (probe 2)

Acetal deprotection was carried out in the same way in accordance with the synthesis method of probe 1. Probe 2 was obtained in a quantity of 1.8 mg as a red solid at a yield of 67% (in two steps) using compound 8 (3 mg) and 300 μL of THF and 200 μL and 120 μL of H₂O.

¹H NMR (300 MHz, CDCl₃): δ 2.27 (s, 3H);2.60 (s, 3H); 2.85 (t, J=7.3 Hz, 2H); 3.36 (t, J=7.3 Hz, 2H); 6.16 (s, 1H); 6.29 (d, J=3.7 Hz, 1H); 6.86 (d, J=3.7 Hz, 1H); 7.10 (s, 1H); 7.65 (d, J=8.8 Hz, 2H); 7.80 (d, J=8.8 Hz, 2H); 7.89 (br, 1H); 9.89 (s, 1H). ¹³C NMR (75 MHz, CDCl₃): d 11.4, 15.0, 24.8, 38.0, 117.6, 119.1, 119.2, 120.8, 124.9, 128.3, 131.1, 132.1, 133.4, 143.6, 144.6, 156.0, 161.0, 170.5, 191.0. HRMS (ESI⁺): m/z calcd for C₂₁H₂₀BFN₃O₂[M−F]⁺, 376.16326; found, 376.16131 (−1.95 mmu).

### Synthesis Examples 3 and 4

Probe 3 and probe 4 were synthesized by scheme 2 below.

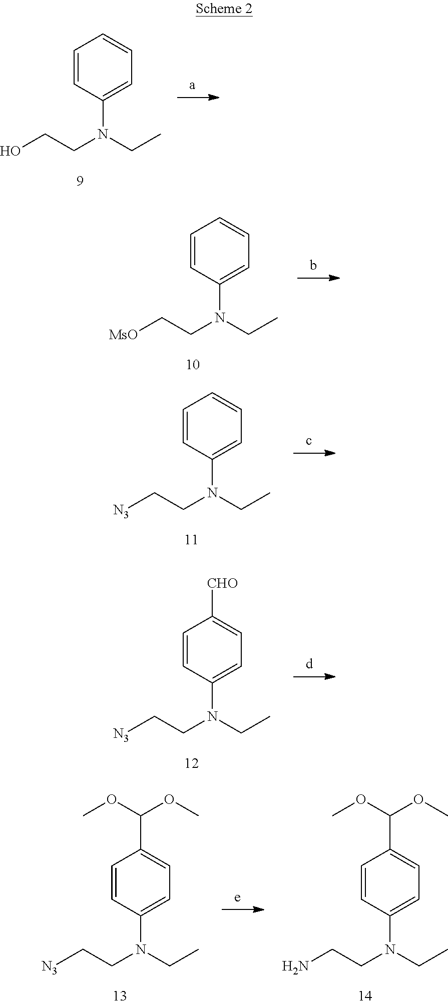 [SMILES: CCN(CCO)c1ccccc1 ; CCN(CCOS(C)(=O)=O)c1ccccc1 ; CCN(CCN=[N+]=[N-])c1ccccc1 ; CCN(CCN=[N+]=[N-])c1ccc(C=O)cc1 ; CCN(CCN=[N+]=[N-])c1ccc(C(OC)OC)cc1 ; CCN(CCN)c1ccc(C(OC)OC)cc1]

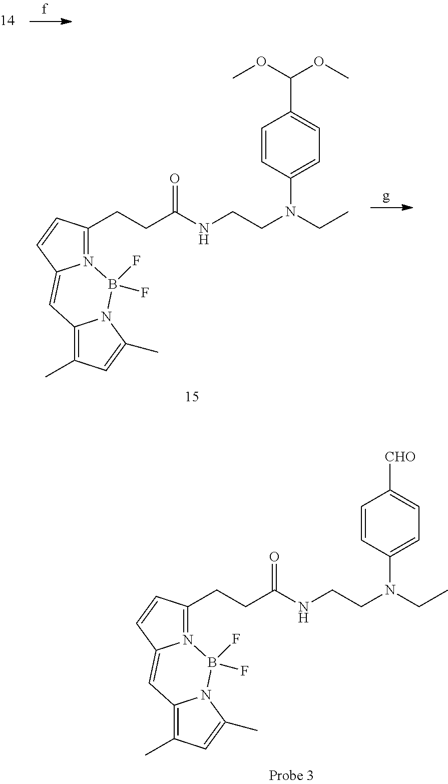

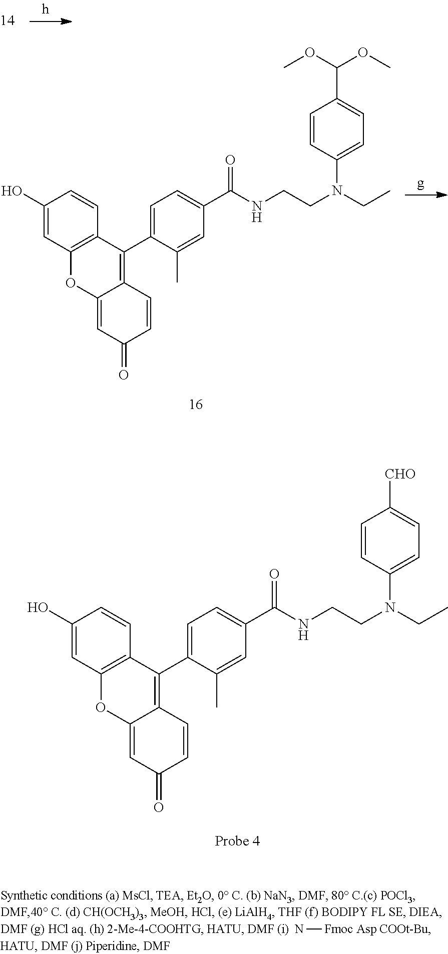

### (1) Synthesis of 2-(N-ethyl-N-(4-dimethoxymethylphenyl)amino)ethyl azide (Compound 13)

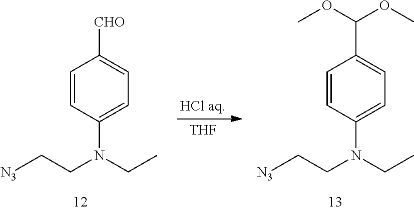 [SMILES: CCN(CCN=[N+]=[N-])c1ccc(C=O)cc1 ; CCN(CCN=[N+]=[N-])c1ccc(C(OC)OC)cc1 ; "THF"]

Compound 12 was synthesized according to the method of the article (Julien Massin, J. et al., “Near-infrared solid-state emitters based on isophorone: Synthesis, crystal structure and spectroscopic properties” Chem. Mater., 2011, 23, pp. 862-873). Compound 12 (730 mg) was weighed out into a 50 mL round-bottomed flask, and 40 mL of distilled MeOH and 1.09 mL of triethyl orthoformate were added. After adding 12 drops of concentrated hydrochloric acid, the solution was heated for three hours at 40° C., then cooled to room temperature. The reaction solution was diluted with MeOH, CH₂Cl₂, sat. NaHCO₃ aq., and the mixed solution was extracted using 20 mL of CH₂Cl₂. The extract was washed using saturated saline, dried using anhydrous sodium sulfate, and filtered. The filtrate was concentrated under reduced pressure and purified by column chromatography using silica gel as the carrier. Compound 12 was obtained in a quantity of 753 mg as a colorless liquid at a yield of 85%.

¹H-NMR (300 MHz, CD₂Cl₂): δ 1.67 (t, J=6.6 Hz, 3H); 3.28 (s, 6H); 3.39-3.65 (m, 6H); 5.26 (s, 1H); 6.69 (d, J=8.8 Hz, 2H); 7.24 (d, J=8.8 Hz, 2H). ¹³C-NMR (75 MHz, CD₂Cl₂): δ 12.3, 45.8, 49.4, 49.9, 52.8, 104.0, 111.9, 1226.6, 128.2, 147.8. HRMS (ESI⁺: m/z calcd for C₁₃H₂₁N₄O₂, [M+H]⁺, 265.16645, found 265.16582 (−0.63 mmu).

### (2) Synthesis of 2-(N-ethyl-N-(4-dimethoxymethylphenyl)amino)ethylamine (Compound 14)

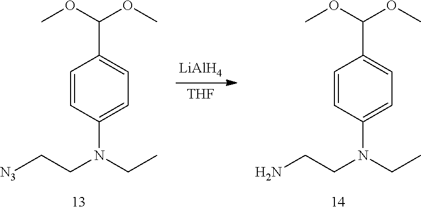 [SMILES: CCN(CCN=[N+]=[N-])c1ccc(C(OC)OC)cc1 ; "LiAlH4" ; CCN(CCN)c1ccc(C(OC)OC)cc1 ; "THF"]

Compound 13 (500 mg, 1.89 mmol) was weighed out into a dried 20 mL round-bottomed flask, and 2 mL of distilled THF was added. After stirring the solution for five minutes on an ice bath, LiAlH₄ (143 mg, 3.78 mmol) suspended in 5 mL of distilled THF was added dropwise while cooling by ice. After dropwise addition, the reaction solution was stirred for 12 hours at room temperature, and 1.5 mL of saturated sodium bicarbonate aqueous solution was added to stop the reaction. Ten milliliters of six shell salt (*1) aqueous solution was added to the suspension. After stirring until a homogeneous solution was produced, the solution was filtered. The filtrate was extracted three times using 30 mL of AcOEt, and the extract was washed using saturated saline, dried using anhydrous sodium carbonate, filtered, and concentrated under reduced pressure. The concentrated residue was purified by column chromatography using NH silica gel as the carrier, and 348 mg of compound 14 was obtained as a colorless liquid at a yield of 77%.

¹H-NMR (300 MHz, CD₂Cl₂): δ 1.43 (t, J=7.3 Hz, 3H); 1.43 (br, 2H); 2.86 (t, J=6.6 Hz, 2H); 3.28 (s, 6H); 3.29-3.45 (m, 6H); 5.25 (s, 1H); 6.78 (d, J=8.8 Hz, 2H); 7.22 (d, J=8.8 Hz, 2H). ¹³C-NMR (75 MHz, CD₂Cl₂): δ12.2, 40.3, 45.7, 52.8, 54.0, 104.1, 111.7, 125.7, 128.0, 148.6. HRMS (ESI⁺): m/z calcd for C₁₃H₂₃N₂O₂, [M+H]⁺, 239.17595; found 239.17701 (+1.06 mmu).

### (3) Synthesis of 2-(N-ethyl-N-(4-formylphenyl)amino)ethyl-3-(1,3-dimethyl-4,4-difluoro-4-bora-3a,4a-diaza-s-indacen-5-yl)-propionamide (Probe 3)

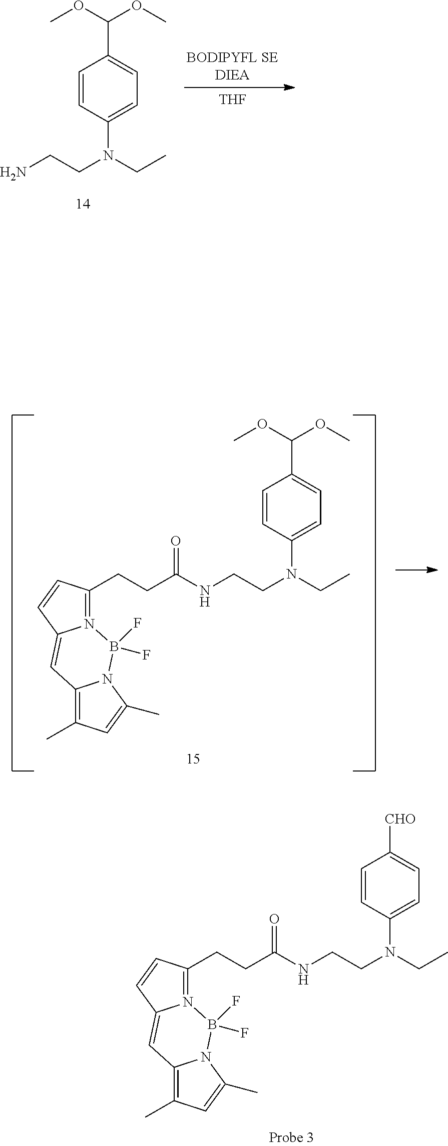

Synthesis was carried out using the same method as compound 4. Compound 14 (11 mg, 39 μmol), BODIPY FL SE (15 mg, 39 μL), and DIEA (7.8 μL, 46 μmol) were used. When purified by column chromatography using silica gel as the carrier, 17 mg of acetal-deprotected probe 3 was obtained as a red solid at a yield of 93%.

¹H-NMR (300 MHz, CDCl₃): δ 1.15 (t, J=7.3 Hz, 3H); 2.26 (s, 3H); 2.55 (s, 3H); 2.65 (d, J=7.3 Hz, 2H); 3.26 (t, J=7.3 Hz, 2H); 3.40 (m, 6H); 6.09 (br, 1H); 6.14 (s, 1H); 6.28 (d, J=3.7 Hz, 1H); 6.70 (d, J=8.8 Hz, 2H); 6.87 (d, J=3.7 Hz, 1H); 7.09 (s, 1H); 7.67 (d, J=8.8 Hz, 2H); 9.68 (s, 1H). ¹³C-NMR (100 MHz, CDCl₃): δ 11.3, 12.1, 15.0, 24.8, 35.9, 37.2, 45.3, 49.1, 110.9, 117.4, 120.6, 123.8, 125.3, 128.2, 132.2, 133.3, 135.3, 144.3, 152.4, 156.8, 160.7, 172.2, 190.1. HRMS (ESI⁺): m/z calcd for C₂₅H₂₉BFN₄O₂, [M−F]⁺, 447.23676; found 447.23230 (−4.46 mmu).

### (4) Synthesis of 2-(N-ethyl-N-(4-formylphenyl)amino)ethyl-3-methyl-4-(6-hydroxyxanthen-3-on-9-yl)-benzamide (Probe 4)

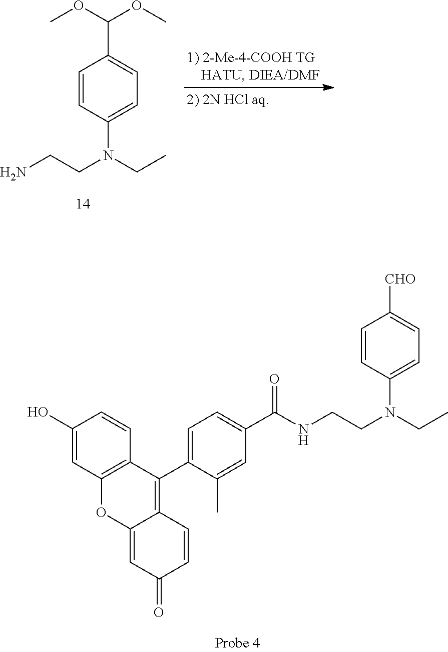

2-Me-4-COOH TG (10 mg, 29 μmol) and compound 14 (8.3 mg, 47 μmol) were weighed out into a 2 mL glass vial, and 270 μL of DMF was added. HATU (13.2 mg, 35 μmol) and 15 μL of DIEA were added to this solution and stirred overnight shielded from light at room temperature in an argon atmosphere. After adding 270 μL of 2N HCl aq. to the reaction solution and stirring for 15 minutes at room temperature, the solution the reaction solution was diluted using an elution solvent for preparative HPLC (A/B=6/4, A: 0.1 M triethylamine acetate buffer; B: A/acetonitrile=2/8). The diluted solution was then purified by preparative HPLC, and the fraction containing the target compound was acidified by 2N HCl aq., then diluted using saturated saline. The diluted solution was extracted three times using 50 mL of AcOEt. The organic layer was then washed by saturated saline, dried using anhydrous sodium sulfate, filtered, and the filtrate concentrated under reduced pressure. Probe 4 was obtained in a quantity of 7.5 mg as a yellow solid at a yield of 50%.

¹H-NMR (300 MHz, CD₃OD): δ 1.24 (t, J=8.0 Hz, 3H); 2.08 (s, 3H); 3.58-3.73 (m, 6H); 6.73 (m, 4H); 6.98 (m, 4H); 7.32 (d, J=7.4 Hz, 1H); 7.71 (d, J=8.8 Hz, 2H); 7.80 (m, 2H); 9.58 (s, 1H). HRMS (ESI⁺): m/z calcd for C₂₉H₃₁NO₈, [M+H]⁺, 521.20497; found 521.20133 (−3.64 mmu).

### Synthesis Example 5

Probe 5 was synthesized according to scheme 3 below.

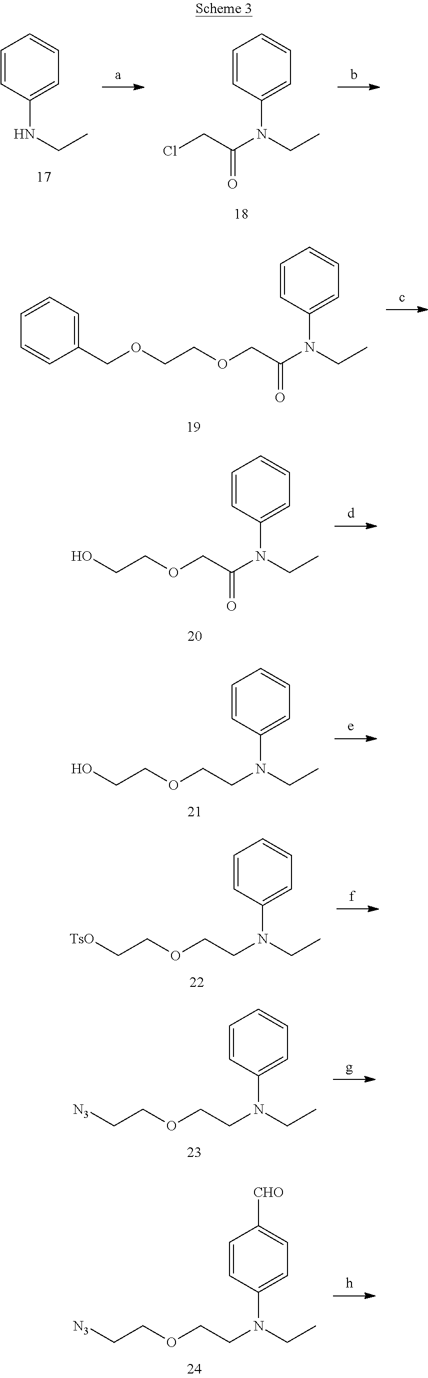

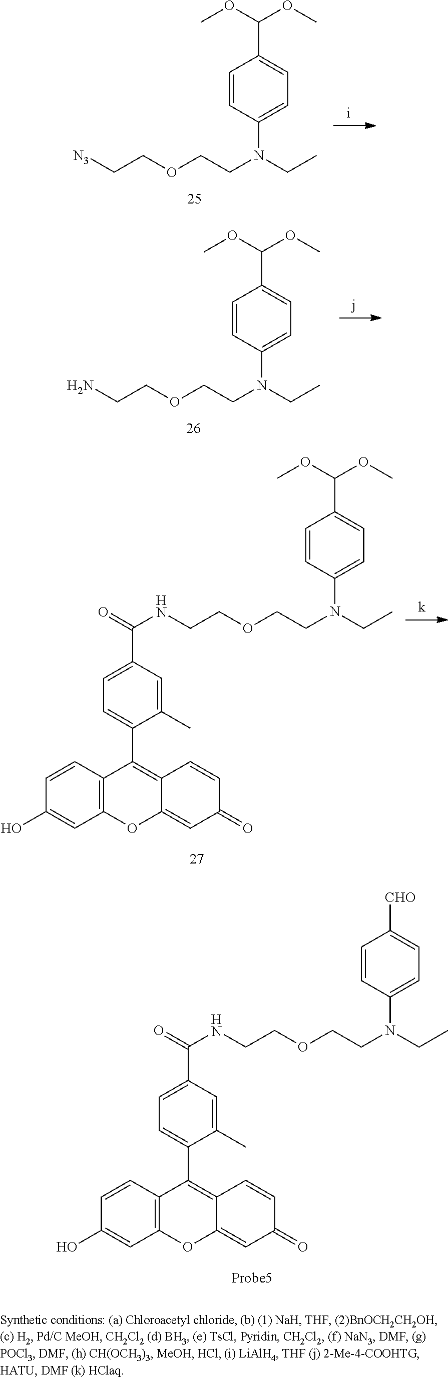 [SMILES: CCN(CCOCCN=[N+]=[N-])c1ccc(C(OC)OC)cc1 ; CCN(CCOCCN)c1ccc(C(OC)OC)cc1 ; CCN(CCOCCNC(=O)c1ccc(-c2c3ccc(=O)cc-3oc3cc(O)ccc23)c(C)c1)c1ccc(C(OC)OC)cc1 ; CCN(CCOCCNC(=O)c1ccc(-c2c3ccc(=O)cc-3oc3cc(O)ccc23)c(C)c1)c1ccc(C=O)cc1]

### (1) Synthesis of N-ethyl-2-(2-benzyloxyethoxy)-N-phenylacetamide (Compound 19)

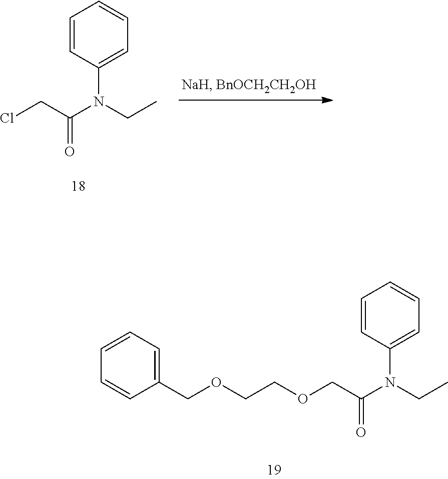 [SMILES: CCN(C(=O)CCl)c1ccccc1 ; CCN(C(=O)COCCOCc1ccccc1)c1ccccc1]

Compound 18 was synthesized according to the method of the article (Lorella Pasquinucci et al., “Evaluation of N-substitution in 6,7-benzomorphan compounds” Bioorg. Med. Chem., 2010, 18, pp. 4975-4982). 2-Benzyloxyethanol (4 g, 26.2 mmol) was weighed out into a dried 100 mL round-bottomed two-necked flask and dissolved in 20 mL of distilled THF. The inside of the container was then replaced with an argon atmosphere using a three-way cock fitted with a balloon filled with argon, and the container was sealed using a rubber septum. A quantity of 1.19 g of NaH (60% dispersion in mineral oil) was added to this solution, stirred for 30 minutes at room temperature in an argon atmosphere, and a solution of sodium 2-benzyloxyethoxide was prepared. Three milliliters of compound 17 was weighed out into a separately prepared round-bottomed flask and dissolved in distilled THF. The sodium 2-benzyloxyethoxide solution prepared above was added using a syringe and stirred for one hour at room temperature. Fifty milliliters of ice water was added gradually to stop the reaction, and the mixed solution was extracted three times using 30 mL of AcOEt. After washing with saturated saline, the extract was dried using anhydrous sodium sulfate, filtered, and concentrated under reduced pressure. The concentrated residue was purified by column chromatography using silica gel as the carrier, and 7.04 g of compound 19 was obtained as a colorless liquid at a yield of 94%.

¹H-NMR (300 MHz, CD₂Cl₂): δ 1.10 (t, J=7.3 Hz, 3H); 3.58 (s, 4H); 3.71 (q, J=7.3 Hz, 2H); 3.82 (s, 2H); 4.49 (s, 2H); 7.15-7.18 (m,2H); 7.27-7.41 (m, 8H). ¹³C-NMR (75 MHz, CD₂Cl₂): δ 13.1, 44.3, 69.8, 70.1, 70.9, 73.4, 127.8, 128.0, 128.5, 128.6, 128.7 .130.0, 138.9, 141.3, 168.6. HRMS (ESI⁺): m/z calcd for C₁₉H₂₃NNaO₃, [M+Na]⁺, 336.15756; found 336.15517 (−2.39 mmu).

### (2) Synthesis of N-ethyl-2-(2-hydroxyethoxy)-N-phenylacetamide (Compound 20)

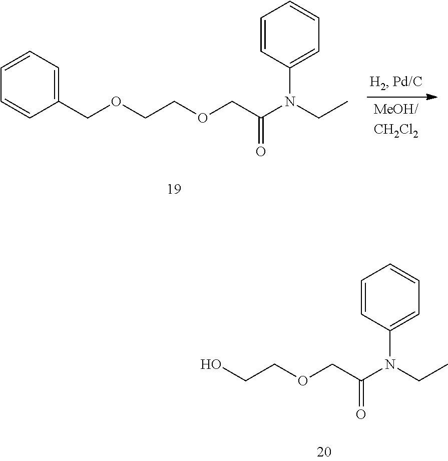 [SMILES: CCN(C(=O)COCCOCc1ccccc1)c1ccccc1 ; CCN(C(=O)COCCO)c1ccccc1]

Compound 19 (3 g, 9.58 mmol) was weighed out into a 200 mL round-bottomed flask and dissolved in 150 mL of distilled MeOH. Three grams of 10% Pd/C was suspended in the solution and stirred at room temperature in a hydrogen atmosphere. The advance of the reaction was confirmed by TLC, and the reaction solution was filtered after the raw materials had disappeared. The filtrate obtained was concentrated under reduced pressure, and 2.05 g of compound 20 was obtained as a light yellow liquid at a yield of 96%.

¹H-NMR (300 MHz, CDCl₃): δ 1.13 (t, 3H); 3.60 (t, J=4.0 Hz, 2H); 3.69 (t, J=4.0 Hz, 2H); 3.72-3.80 (m, 3H); 3.84 (s, 2H); 7.15 (d, J=6.6 Hz, 2H); 7.43 (m, 3H). ¹³C-NMR (75 MHz, CDCl₃): δ 12.5, 44.0, 61.2, 68.9, 73.7, 127.8, 128.2, 129.6, 139.9, 169.4. HRMS (ESI⁺): m/z calcd for C12H17NNaO3, [M+Na]+, 246.11061; found 246.11242 (+1.80 mmu).

### (3) Synthesis of 2-(2-(N-ethyl-N-phenylamino)ethoxy)ethanol (Compound 21)

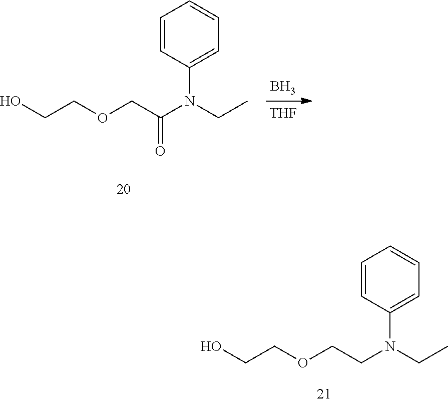

Compound 20 (2.04 g, 9.16 mmol) was weighed out into a dried 100 mL round-bottomed flask and dissolved in 100 mL of distilled THF. After replacing the interior of the flask with an argon atmosphere, 51 mL of BH₃ THF solution (0.9 mmol/mL) was added dropwise at room temperature to the solution. The advance of the reaction was monitored by TLC, and the reaction was stopped by adding distilled water after stirring at room temperature and confirming the disappearance of the raw materials. The reaction solution was concentrated under reduced pressure. The concentrated solution was extracted three times using 50 mL of AcOEt, and the extract was washed with saturated saline, dried using anhydrous sodium sulfate, and filtered. The filtrate obtained was concentrated under reduced pressure, and 1.84 g of compound 21 was obtained as a colorless liquid at a yield of 96%.

¹H-NMR (300 MHz, CDCl₃): δ1.15 (t, J=6.6 Hz, 3H); 3.42 (q, J=6.6 Hz, 2H); 3.50 (t, J=5.8 Hz, 2H); 3.56 (t, J=5.2 Hz, 2H); 3.64 (t, J=5.8 Hz, 2H); 3.71 (t, J=5.1 Hz, 2H); 6.69 (m, 3H); 7.21 (m, 2H). ¹³C-NMR (75 MHz, CDCl₃): δ 12.1, 45.4, 50.0, 61.8. 68.9. 72.3, 112.0 116.0, 129.3, 147.8. HRMS (ESI⁺): m/z calcd for C₁₂H₂₀NO₂, [M+H]⁺, 210.14940; found 210.15041 (+1.01 mmu).

### (4) Synthesis of 2-(2-(N-ethyl-N-phenylamino)ethoxy)ethyl p-toluenesulfonate (Compound 22)

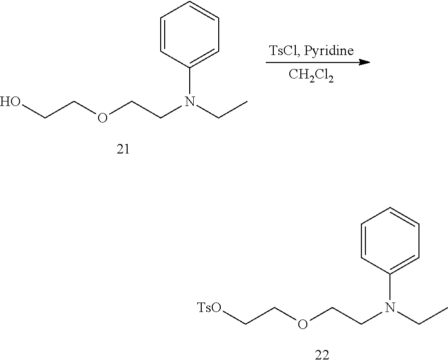 [SMILES: CCN(CCOCCO)c1ccccc1 ; "TsCl, Pyridine" ; "CH2Cl2" ; CCN(CCOCCOS(=O)(=O)c1ccc(C)cc1)c1ccccc1]

Compound 21 (1.84 g, 8.8 mmol) and TsCl (2.52 g, 13.2 mmol) were weighed out into a dried 50 mL round-bottomed flask. The flask was set on an ice bath, and 30 mL of distilled CH₂Cl₂ was added. After adding pyridine (1.06 mL, 13.2 mmol) dropwise to this reaction solution while cooling by ice, the reaction solution was stirred overnight at room temperature. After adding 30 mL of distilled water to the reaction solution obtained, the mixed solution was extracted twice using 30 mL of CH₂Cl₂. The extract was washed with saturated saline, dried using anhydrous sodium sulfate, and filtered. The filtrate obtained was concentrated under reduced pressure. The residue was purified by column chromatography using silica gel as the carrier, and 6.15 g of compound 22 was obtained at a yield of 70%. The compound 22 obtained was unstable and was immediately used in the next reaction.

¹H-NMR (300 MHz, CDCl₃): δ1.11 (t, J=7.3 Hz, 3H); 2.43 (s, 3H); 3.35 (q, J=7.3 Hz, 2H); 3.42 (t, J=5.9 Hz, 2H); 3.56 (t, J=5.8 Hz, 2H); 4.15 (t, J=4.3 Hz, 2H); 6.65 (m, 3H); 7.19 (m, 2H); 7.33 (d, J=8.1 Hz, 2H); 7.79 (d, J=8.1 Hz, 2H). HRMS (ESI⁺): m/z calcd for C₁₉H₂₆NO₄S, [M+H]⁺, 364.15825; found 364.15403 (−4.23 mmu).

### (5) Synthesis of 2-(2-(N-ethyl-N-phenylamino)ethoxy)ethyl azide (Compound 23)

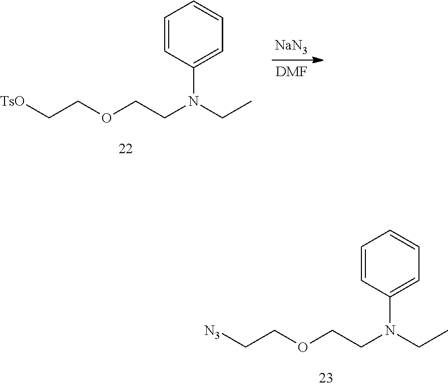

Compound 22 (2.21 g, 6.09 mmol) and NaN₃ (474 mg, 7.3 mmol) were added to a 50 mL round-bottomed flask, and 10 mL of DMF was added. The reaction solution was heated to 90° C. and stirred overnight. The reaction solution obtained was diluted using 50 mL of distilled water and extracted three times using 30 mL of AcOEt. The extract was washed twice with 20 mL of distilled water and once with saturated saline, dried using anhydrous sodium sulfate, and filtered. The filtrate obtained was concentrated under reduced pressure. The concentrated residue was purified by column chromatography using NH silica gel as the carrier, and compound 23 was obtained.

¹H-NMR (300 MHz, CDCl₃): δ1.16 (t, J=7.5 Hz, 3H); 3.36 (t, J=5.1 Hz, 2H); 3.42 (q, J=7.5 Hz, 2H); 3.51 (t, J=5.9 Hz, 2H); 3.64 (m, 4H); 6.63-6.70 (m, 3H); 7.18-7.24 (m, 2H). ¹³C-NMR (75 MHz, CDCl₃): δ 12.1, 45.4, 50.0, 50.8, 69.1, 70.1, 111.8, 115.8.128.9, 129.3, 147.7. HRMS (ESI⁺): m/z calcd for C₁₂H₁₀N₄O, [M+H]⁺, 235.15589; found 235.15256 (−3.32 mmu).

### (6) Synthesis of 2-(2-(N-ethyl-N-(4-formylphenyl)amino)ethoxy)ethyl azide (Compound 24)

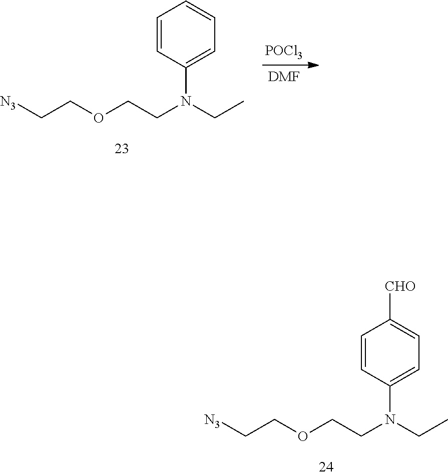

Compound 23 (1.21 g, 5.18 mmol) was weighed out into a dried 50 mL round-bottomed flask and dissolved in 5 mL of DMF. The flask was stirred on an ice bath for five minutes, and POCl₃ (966 mL, 10.37 mmol) was then added dropwise. The reaction solution was heated and stirred overnight at 40° C. thereafter. The flask was then cooled on an ice bath, and 50 mL of NaHCO₃ was added. The solution obtained was extracted three times using 30 mL of AcOEt. The extract was dried using anhydrous sodium sulfate, filtered, and concentrated under reduced pressure. The concentrated residue was purified by column chromatography using silica gel as the carrier, and 1.03 g of compound 24 was obtained as a pale green liquid at a yield of 76%.

¹H-NMR (300 MHz, CDCl₃): δ1.21 (t, J=7.3 Hz, 3H); 3.37 (t, J=4.4 Hz, 2H); 3.55 (q, J=7.3 Hz, 2H); 3.60-3.70 (m, 6H); 6.72 (d, J=9.5 Hz, 2H); 7.71 (d, J=8.9 Hz, 2H); 9.73 (s, 1H). ¹³C-NMR (75 MHz, CDCl₃): δ 12.0, 45.8, 50.1, 50.7, 68.8, 70.2, 110.8, 125.1, 132.2, 152.3, 190.0. HRMS (ESI⁺): m/z calcd for C₁₃H₁₈N₄NaO₂, [M+Na]⁺, 285.13274; found 285.13462 (−1.87 mmu).

### (7) Synthesis of 2-(2-(N-ethyl-N-(4-dimethoxymethylphenyl)amino)ethoxy)ethyl azide (Compound 25)

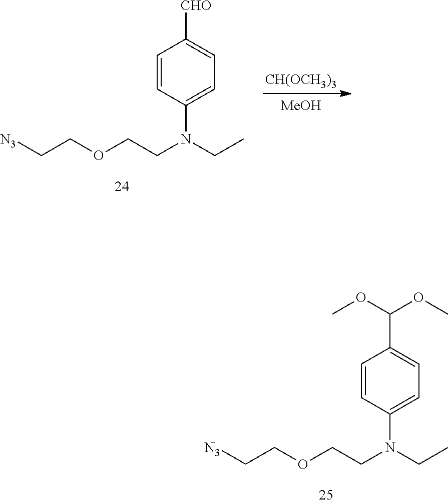

Acetal protection of compound 24 was carried out using the method shown in the synthesis of compound 13. Compound 25 was obtained as a light brown liquid at a yield of 100% using compound 24 (1.16 g, 6.33 mmol), trimethyl orthoformate (2.08 mL, 19.0 mmol), and c. HCl (10 drops).

¹H-NMR (300 MHz, CDCl₃): δ1.15 (t, J=7.3 Hz, 3H); 3.31 (s, 6H); 3.33-3.65 (m, 10H); 5.28 (s, 1H); 6.65 (d, J=8.8 Hz, 2H); 7.20 (d, J=8.8 Hz, 2H). ¹³C-NMR (75 MHz, CDCl₃): δ 12.0, 45.4, 49.7, 50.0, 50.7 .52.6, 52.8, 53.8, 68.9, 70.0, 103.6, 110.8, 111.1, 125.1, 127.6, 147.7. HRMS (ESI⁺): m/z calcd for C₁₅H₂₄N₄NaO₃, [M+Na]⁺, 331.17461; found 331.17097 (−3.64 mmu).

### (8) Synthesis of 2-(2-(N-ethyl-N-(4-dimethoxymethylphenyl)amino)ethoxy)ethylamine (Compound 26)

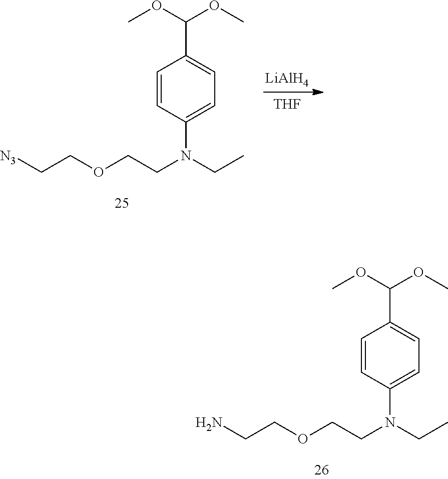 [SMILES: CCN(CCOCCN=[N+]=[N-])c1ccc(C(OC)OC)cc1 ; "LiAlH4" ; CCN(CCOCCN)c1ccc(C(OC)OC)cc1 ; "THF"]

Azide group reduction of compound 25 was synthesized according to the synthesis method of compound 14 shown above. Compound 26 was obtained in a quantity of 629 mg as a colorless liquid at a yield of 83% using compound 25 (823 mg, 2.67 mmol), LiAlH₄ (203 mg, 5.34 mmol), and 10 mL of THF.

¹H-NMR (300 MHz, CD₂Cl₂): δ1.14 (t, J=7.3 Hz, 3H); 1.41 (br, 2H); 2.77 (t, J=5.9 Hz, 2H); 3.26 (s, 6H); 3.40-3.50 (m, 6H); 3.59 (t, J=6.6 Hz, 2H); 5.23 (s, 1H); 6.66 (d, J=8.8 Hz, 2H); 7.20 (d, J=8.8 Hz, 2H). ¹³C-NMR (75 MHz, CD₂Cl₂): δ 12.3, 42.3, 45.7, 50.5, 52.8, 69.0, 74.1, 104.1, 111.4, 125.7, 128.0, 148.4. HRMS (ESI⁺): m/z calcd for C₁₄H₂₃N₂O₂, [M−OCH₃]⁺, 251.1795; found 251.17451 (−1.45 mmu).

### (9) Synthesis of 2-(2-(N-ethyl-N-(4-formylphenyl)amino)ethoxy)ethyl-3-methyl-4-(6-hydroxyxanthen-3-on-9-yl)-benzamide (Probe 5)

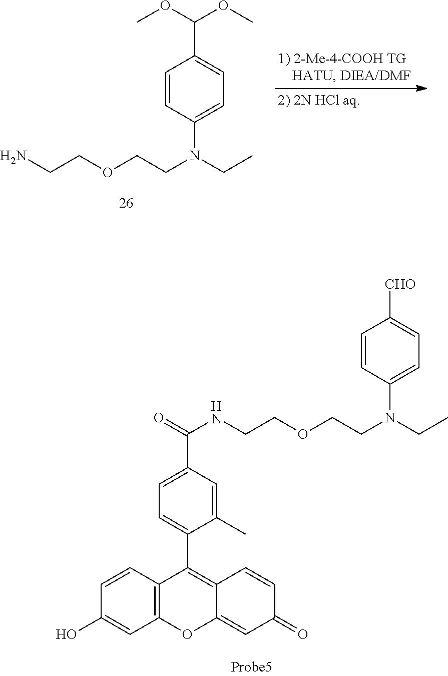

Compound 26 was synthesized according to the synthesis method of probe 4 shown above. Probe 5 was obtained in a quantity of 15 mg as an orange solid at a yield of 49% using compound 29 (21.4 mg, 75 μmol), 2-Me 4-COOH TG (20 mg, 58 μmol), HATU (26 mg, 69 μmol), and DIEA (22 mL, 173 μmol).

¹H-NMR (300 MHz, CD₃OD): δ 1.19 (t, J=7.3 Hz, 3H); 2.11 (s, 3H); 3.56-3.73 (m, 11H); 6.70-6.73 (m, 4H); 6.83 (d, J=8.8 Hz, 2H); 7.32 (d, J=8.0 Hz, 1H); 7.65 (d, J=8.8 Hz, 2H); 7.79 (d, 6.6 Hz, 1H); 7.87 (br, 1H); 9.53 (s, 1H). HRMS (ESI⁺): m/z calcd for C₃₄H₃₃N₂O₆, [M+H]⁺, 565.2386; found 565.23793.

### Reference Examples 1-3

The reactivity of Probes 1-3 synthesized as described above to ALDH1A1, ALDH1A3, and ALDH3A1 was investigated.

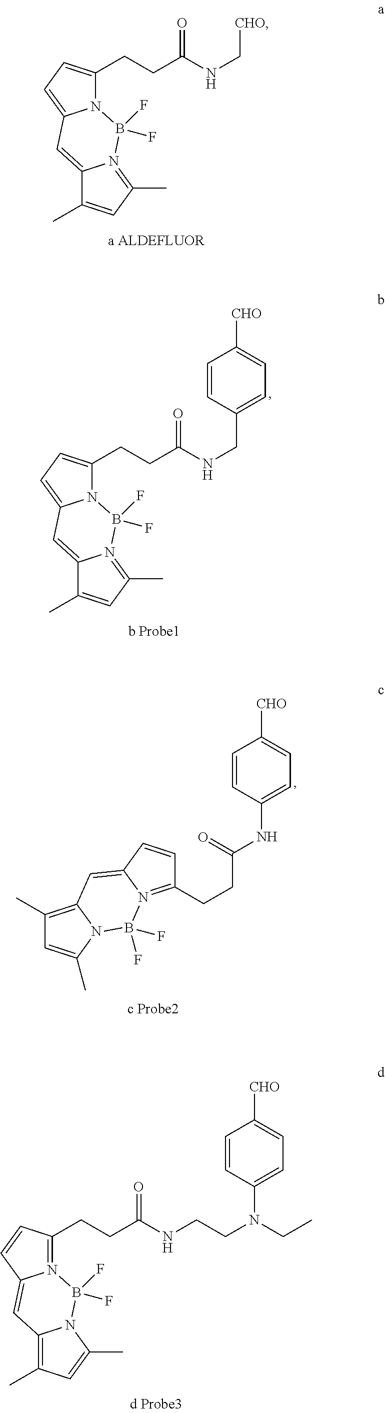

Human recombinant ALDH1A1/1A3/3A1 was purchased, and 100 mM of KCL, 2 mM of DTT, 1 mM of NAD(P), 10-100 nM of each ALDH, and 10-80 μM of each compound were mixed in Tris buffer (100 mM, pH 7.5), reacted for 30 minutes at 37° C., an equal amount of acetonitrile added, and the reaction stopped. The reaction solution was analyzed by UPLC/MS/MS (Waters). The UPCL chromatogram was carried out over five minutes at a flow rate of 800 μL/min by a linear gradient from 5% acetonitrile 0.01 M ammonium formate/water to 95% acetonitrile 0.01 M ammonium formate/water. An aldehyde-form and carboxylic acid-form probe were detected by the absorbance at a wavelength of 504 nm, and m/z of the MS spectrum at each peak was confirmed to agree with the expected m/z.

Also, since ALDEFLUOR is known to react with ALDH1A1 and ALDH1A3, the study was conducted only for ALDH3A1. The results are shown in Table 2.

Compound 1 which has benzaldehyde as the reaction site reacted with all three of the isoforms tested, but compound 2 presented reactivity only with ALDH1A1. On the other hand, compound 3 which has DEAB as the reaction site reacted with ALDH1A1 and ALDH3A1, and the results were the most specific for ADLH3A1 among the three probes. Also, as expected, ALDEFLUOR showed no reactivity to ALDH3A1.

TABLE 2
Reactivity of probes 1-3 to each ALDH isoform

| | Isoform | | |
|---|---|---|---|
| Probe | ALDH1A1 | ALDH1A3 | ALDH3A1 |
| Probe 1 | ◯ | ◯ | ◯ |
| Probe 2 | ◯ | X | X |
| Probe 3 | ◯ | X | ◯ |
| ALDEFLUOR | ◯ | ◯ | X |

Cell imaging was carried out using probe 1 and probe 3 which showed reactivity to ALDH3A1. An esophageal squamous cell carcinoma cell line (OE21 cell line) which are cells that express a high level of ALDH3A1 was used as the cells. Imaging was attempted with various probe concentrations and reaction times, but no evident difference in brightness could be found in comparison to the negative control which used an ALDH3A1-specific inhibitor (CB7). Also, thinking that probe 2 could be used in the same way as ALDEFLUOR which is capable of detecting cells having high ALDH1 activity, imaging was carried out using A549 cells since ALDEFLUOR is reported in the literature to be able to detect cells having high ALDH1A1 activity. Nonetheless, no evident difference in brightness was found in comparison to the negative control.

### Example 1 and Reference Example 4

Next, the reactivity of probes 4 and 5 synthesized as described above to ALDH1A1, ALDH1A3, and ALDH3A1 was investigated.

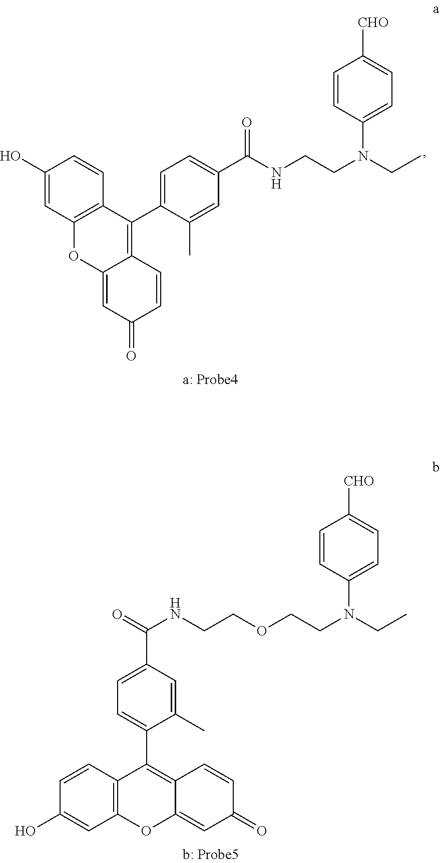 [SMILES: CCN(CCNC(=O)c1ccc(-c2c3ccc(=O)cc-3oc3cc(O)ccc23)c(C)c1)c1ccc(C=O)cc1 ; CCN(CCOCCNC(=O)c1ccc(-c2c3ccc(=O)cc-3oc3cc(O)ccc23)c(C)c1)c1ccc(C=O)cc1]

The reason why the intended results were not obtained for probes 1-3 was thought to be excessive membrane permeability. BODIPY is a hydrophobic probe, but probes 1-3 are strongly hydrophobic aromatic aldehydes different from the short-chain aliphatic aldehyde contained in the reaction site of ALDEFLUOR. In short, the combination of hydrophobic compounds is thought to shift the water solubility of the probe as a whole to the hydrophobic side, and even when the probe becomes a carboxylic acid form, the hydrophilicity is not enough to make the probe membrane-impermeable. Therefore, it was decided to use a fluorophore more hydrophilic than BODIPY to make the probe more hydrophilic, and Tokyo Green (TG) was used as the fluorophore. It was decided to use DEAB which has relatively high specificity for ALDH3A1 for the reaction site. Also, since the binding site also affects the reactivity to ALDH according to the results of probes 1 and 2, linkers with two different length binding sites were used in probes 4 and 5.

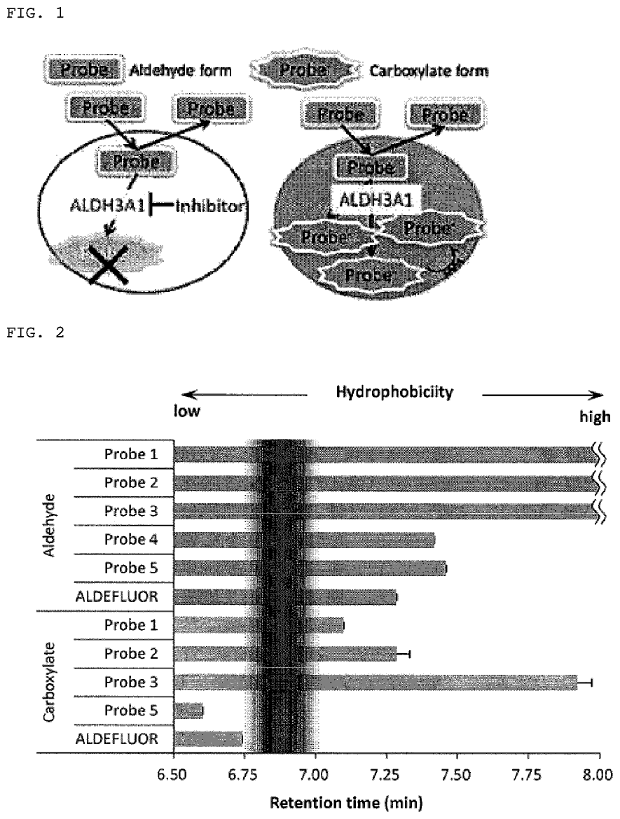

When the reactivity to ALDH was checked, probe 4 presented absolutely no reactivity, but probe 5, like probe 3, showed reactivity to ALDH1A1 and ALDH3A1. The water solubility was verified by LC/MS under neutral conditions to compare the membrane permeability of the five probes produced above and ALDEFLUOR. The reaction was stopped after advancing the reaction to the point that each probe was present as both a carboxylic acid form and an aldehyde form using a suitable isoform and concentration of ALDH, and analysis was conducted by LC/MS (Agilent 1200/6130 quadrupole LC/MS system). A C18 column (HP 3 μm, inner diameter: 2.1 mm, length: 150 mm, GL Science) was used as the column. HPLC chromatograms were carried out by 20% B for 2.5 minutes followed by a five-minute linear gradient (flow rate: 500 μL/min) from 20% to 100% B, taking solvent A to be 0.01 M ammonium formate/water and solvent B to be 80% acetonitrile 0.01 M ammonium formate/water. The probe containing BODIPY was detected by the absorbance at a wavelength of 504 nm and the probe containing TG at a wavelength of 495 nm, and m/z of the MS spectrum at each peak was confirmed to agree with the expected m/z. Three independent tests were run, and the mean±SD was calculated. The data obtained are shown in Table 3 and FIG. 2. Aldehyde-form probes of compounds 1, 2, and 3 were detected after 8 minutes, but aldehyde-form probes 4 and 5 and ALDEFLUOR were detected in the lower 7 minutes. On the other hand, probe 1 which had the earliest detection time of the carboxylic acid-form probes 1, 2, and 3 was detected at 7.098 minutes, but carboxylic acid-form ALDEFLUOR was 6.741 minutes, and carboxylic acid-form probe 5 was detected even earlier than that. Given that imaging of probe 1 was not successful, there was thought to be a threshold at which the membrane permeability changes rapidly between this 6.741 minutes and 7.098 minutes (band in FIG. 2).

TABLE 2
Reaction of probes 4 and 5 to each ALDH

| | Isoform | | |
|---|---|---|---|
| Probe | ALDH1A1 | ALDH1A33 | ALDH3A1 |
| Probe 4 | X | X | X |
| Probe 5 | ◯ | X | ◯ |

TABLE 3
Retention times of the aldehyde form and
carboxylic acid form of each probe

Retention Time (min)

| Probe | Aldehyde | carboxylate |
|---|---|---|
| Probe 1 | 8.874 ± 0.000 | 7.098 ± 0.001 |
| Probe 2 | 9.330 ± 0.002 | 7.284 ± 0.047 |
| Probe 3 | 9.153 ± 0.010 | 7.920 ± 0.052 |
| Probe 4 | 7.418 ± 0.001 | N.D. |
| Probe 5 | 7.459 ± 0.003 | 6.602 ± 0.001 |
| ALDELFLUOR ® | 7.283 ± 0.003 | 6.741 ± 0.001 |

(HPLC: A 0.01M AF/water, B 80% MeCN 0.01M AF/water 20% B 2.5 min; 20%-->100% B 5 min)

### (Kinetic Characteristics of Probes)

Km and Kcat in the Michaelis-Menten equation were determined in each probe for each ALDH isoform that showed reactivity. For the reaction, 100 mM of KCL, 2 mM of DTT, 1 mM of NAD(P), and five to six concentrations within the range of 10-100 nM of each ALDH and 0.1-80 μM of each probe were selected, mixed in Tris buffer (100 mM, pH 7.5), and reacted for five minutes at 37° C.; an equal amount of acetonitrile was added, and the reaction was stopped. Detection of the aldehyde-form/carboxylic acid-form probes was carried out using the same method as described above by UPLC/MS/MS. Km and Kcat were calculated by fitting the reaction rate at each probe concentration obtained to the Michaelis-Menten equation. A Kaleida Graph ver. 4.1 (Hulinks, Inc.) was used in fitting. The data obtained are shown in Table 4.

The Kcat ratio (Kcat of ALDH3A1/Kcat of ALDH1A1) was calculated for probe 1, probe 3, and probe 5 as an indicator of ALDH1A1/ALDH3A1 specificity because the probe reaction rate at the actual probe use concentration is believed to be close to Vmax since Km is relatively small (Table 5). Probe 5 had approximately six times higher reactivity with ALDH3A1 than with ALDH1A1 at the same ALDH concentration and at a probe concentration where the reaction rate is close to Vmax and was a probe with higher specificity for ALDH3A1.

TABLE 4
Kinetic characteristics of each probe

| | ALDH1A1 | | ALDH1A3 | | ALDH3A1 | |
|---|---|---|---|---|---|---|
| | Kₘ | K_{cat} | Kₘ | K_{cat} | Kₘ | K_{cat} |
| | (μM) | (min⁻¹) | (μM) | (min⁻¹) | (μM) | (min⁻¹) |
| Probe 1 | <0.2 | 81.5 ± 2.7 | 1.64 ± 0.49 | 1.74 ± 0.09 | 58.9 ± 7.6 | 67.2 ± 4.8 |
| Probe 2 | 5.76 ± 2.39 | 204 ± 24.9 | — | — | — | — |
| Probe 3 | 0.17 ± 0.03 | 6.05 ± 0.20 | — | — | 0.87 ± 0.07 | 12.2 ± 0.2 |
| Probe 4 | — | — | — | — | — | — |
| Probe 5 | 0.26 ± 0.05 | 1.02 ± 0.04 | — | — | 3.49 ± 0.55 | 5.97 ± 0.26 |
| ALDEFLUOR ® | 161 ± 144 | 465 ± 377 | N.E. | N.E. | — | — |

N.E.: not examined

TABLE 5
Kcat ratio (Kcat of ALDH3A1/Kcat of ALDH1A1) of probes 1, 3, and 5
Kcat ratio

| | Kcat^{ratio} (ALDH3A1/ALDH1A1) |
|---|---|
| Probe 1 | 0.8 |
| Probe 3 | 2 |
| Probe 5 | 5.9 |

### Example 2

### (Cell Imaging Using Probe 5)

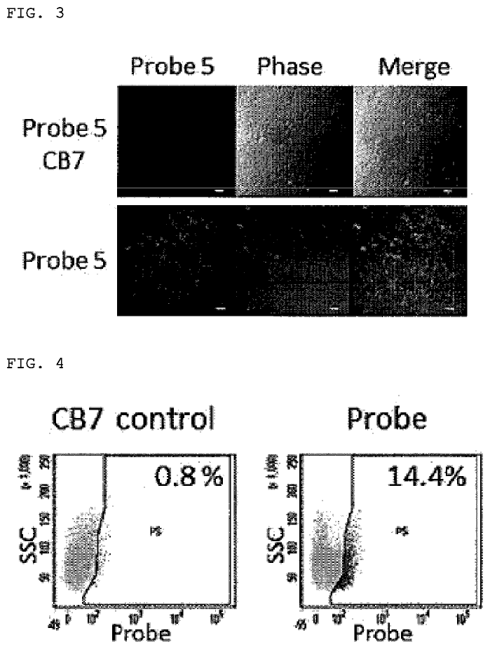

Imaging was carried out using probe 5 and an esophageal squamous cell carcinoma cell line (0E21 cell line) which are cells that express a high level of ALDH3A1. 40 μM of probe 5 was added to culture medium (RPMI 1640, 10% FBS, 15 mM HEPES, phenol red-free). After culturing for 90 minutes at 37° C., the cells were washed using a chilled medium and examined under a confocal microscope. A sample with a specific ALDH3A1 inhibitor (1-[(4-fluorophenyl)sulfonyl]-2-methyl-1H-benzimidazole, CB7, 10 μM) added served as a negative control. Excitation was provided by a 488 nm laser, and observation was carried out at wavelengths of 500-570 mm in detection. As shown in FIG. 3, high-brightness cells could be observed in comparison to the negative control.

### Example 3

### (Flow Cytometry Using Probe 5)

Flow cytometry was carried out using probe 5 and OE21 cells. The cells were treated using trypsin, and the cell density was measured after passing the cells through a cell strainer (40 μm, Corning). A quantity of 500 μL of a cell suspension to make 2.5-5×10⁵/mL in medium containing 40 μM of probe 5 was prepared and cultured for 90 minutes at 37° C. After centrifuging for three minutes at 1500 rpm and removing the supernatant after the reaction, the cells were washed with chilled medium, centrifuged, and the medium was removed. The cells were again suspended in 100 μL of medium, and 4 μL of SYTOX (registered trademark) Red Dead Cell Stain (Thermo Fisher) was added. After standing for five minutes on ice, 150 μL of medium was added. A sample with CB7 (10 μM) added was used as a negative control. Analysis was carried out using FACS Canto II (BD Biosciences). Excitation was provided by a blue laser (488 nm), and the brightness of probe 5 was detected in each cell by FITC channel (515-545 nm). About 30,000 cells were analyzed in each sample after removing doublets and removing dead cells. The positive gate was set in a range from near to above the upper limit of brightness in the negative control according to ALDEFLUOR assay, so that at most 3% of the cells were present within the gate in the negative control sample. Three independent tests were run. The positive rate was 10-15%, as shown in FIG. 4.

### Example 4

### (Flow Cytometry by ALDH3A1 Knockdown)

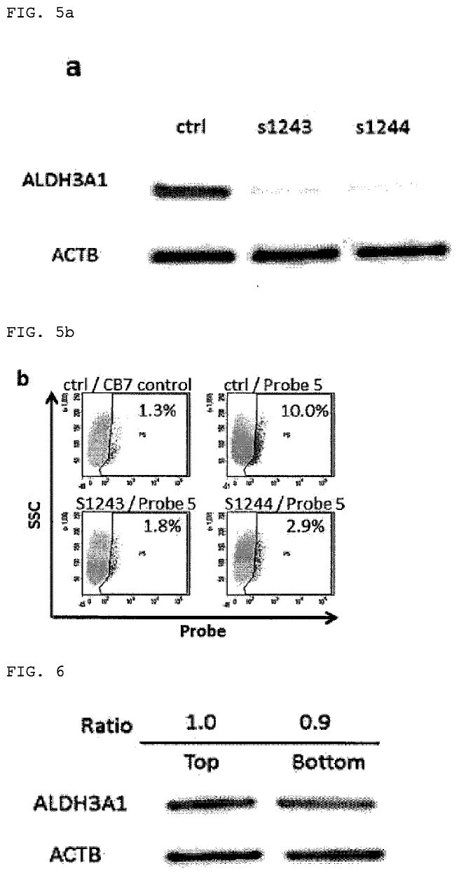

ALDH3A1 was knocked down using small interference RNA (siRNA) to confirm that the positive group in flow cytometry using 0E21 cells and compound 5 was due to ALDH3A1 activity, and flow cytometry was conducted. Two siRNAs confirmed to be effective (Silencer (registered trademark) Select, s1243 and s1244) and a negative control (Silencer® negative control #1) were purchased from Thermo Fisher. Lipofectamine® RNAiMAX as transfection reagent and Opti-MEM® as diluent were purchased from Thermo Fisher. According to the instruction manual, 300 μL of siRNA-Lipofectamine complex was produced according to the instruction manual so that siRNA was 10 nM and Lipofectamine was 3%, and 250 μL thereof was mixed with 2.5 mL of OE21 cell suspension and sown in six-well dishes. The effect of knockdown was confirmed after six days by Western blotting (FIG. 6a). When flow cytometry was carried out under the same conditions as described above using probe 5 under these conditions, the positive rate decreased significantly in the ALDH3A1 knockdown group, and the probe 5-positive cells were shown to be due to ALDH3A1 activity (FIG. 5).

### Example 5

### (Confirmation of ALDH3A1 Expression Level by Sort Out)

The activity of ALDH is regulated by the expression level. Conducting Western blotting using antibodies to the ALDH isoforms is common for sort out since the activity of at least ALDH1A1, ALDH1A3, and ALDH2 is detected indiscriminately in ALDEFLUOR assay. Sort out into a high-brightness group and a low-brightness group was therefore carried out using probe 5 and OE21 cells, and Western blotting was conducted. An FACS Aria II (BD Biosciences) was used as the cell sorter. Assay was carried out in the same way as in ordinary flow cytometry, a positive gate (top) and a gate (bottom) for the bottom 10-15% on the low-brightness side were set, and the cells were sorted out.

A lysate was produced from the cells obtained, and Western blotting was conducted. Each band was detected and quantified using an ImageQuant LAS 4000 mini (GE Healthcare) by a luminescent method. The ALDH3A1 expression level was standardized by ACTB which is a housekeeping gene, and the ALDH3A1 expression levels were compared in the top and bottom. Contrary to expectation, there was not that much difference in the expression levels, as shown in FIG. 6.

### Example 6

### (Extracellular Active Discharge of Probe and Optimization by Control Thereof)

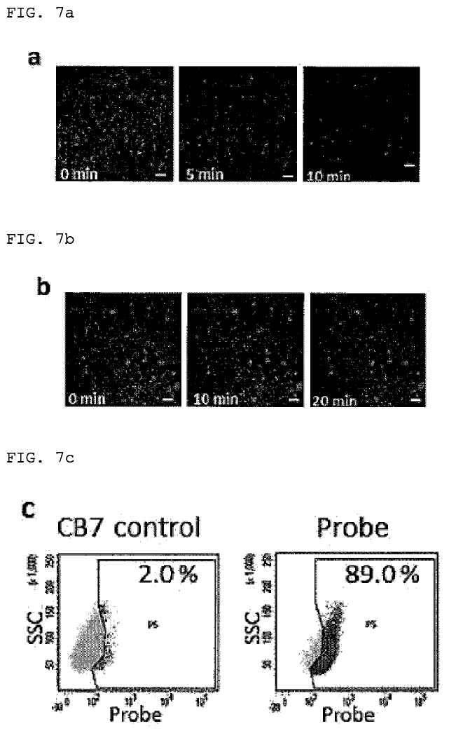

Cells have an active discharge mechanism for substances within the cell. The active discharge function is known to differ depending on the cell even within the same cell line. In consideration of active discharge, chilled medium was used in imaging and flow cytometry, but the same visual field was examined over time at 37° C. to evaluate active discharge (FIG. 7a).

As a result, a pronounced decrease in fluorescent brightness was seen about 10 minutes after the start of observation.

TG is a derivative of fluorescein but is known to be discharged extracellularly by the action of multidrug resistance-associated protein (MRP), which is a fluorescein transporter. Given that this active discharge is reported to be suppressed by MRP inhibitors, whether the decrease in the brightness of compound 5 was suppressed was tested using several MRP inhibitors. MK-571 is a chemical that inhibits multiple isoforms of MRP. Active discharge of probe 5 was markedly suppressed when MK-571 was used in a concentration of 200 μm (FIG. 7b). Marked improvement of the positive rate to about 90% was obtained when flow cytometry was therefore carried out with MK-571 (200 μM) added (FIG. 7c).

### Example 7

### (Flow Cytometry by ALDH3A1 Knockdown Cells Using MK-571 in Combination)

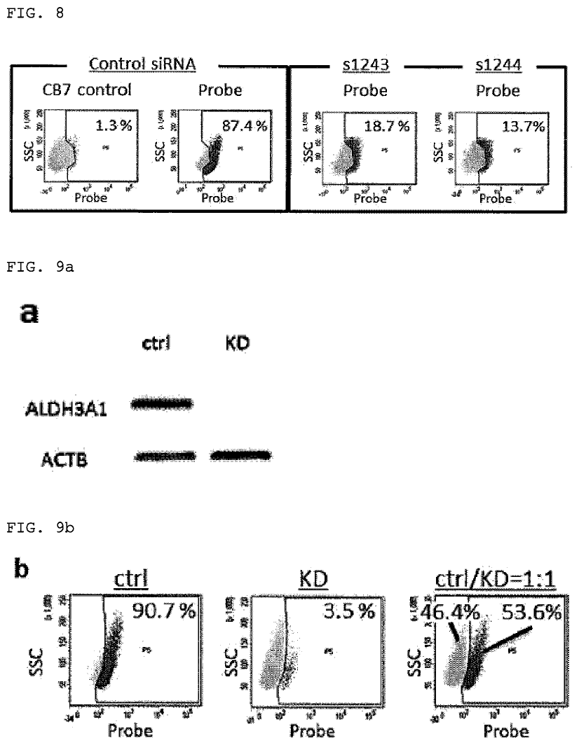

The positive rate improved markedly in flow cytometry using MK-571 in combination, but ALDH3A1 of OE21 cells was knocked down using siRNA and flow cytometry was carried out to confirm that the positive cells were due to ALDH3A1 activity (FIG. 8). When assay was carried out using 200 μm of MK-571, 50 μm of probe 5, and 20 μM of the negative control CB7, the positive rate in negative control cells treated by siRNA was about 90% but decreased markedly to around 10% due to knockdown, which confirmed that the positive cells in assay in which the positive rate was markedly improved using MK-571 derived from ALDH3A1 activity.

### Example 8

### (Separation and Sorting Out of Positive Cells/Negative Cells)

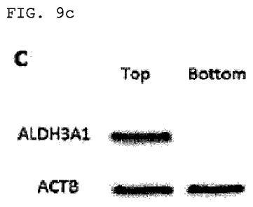

Cells in which ALDH3A1 was knocked down permanently were produced by short hairpin RNA (shRNA) to show that positive cells and negative cells can be separated and sorted out. shRNA against ALDH3A and non-target shRNA as a negative control were introduced into OE21 cells using a lentivirus. Lysates were produced using negative control cells (ctrl) in which ALDH3A1 was not knocked down and cells (KD) in which ALDH3A1 was permanently knocked down. Western blotting was carried out, and pronounced knockdown of ALDH3A1 was confirmed (FIG. 9a). When flow cytometry using MK-571 and probe 5 was carried out by the same protocol as described above using these cells (FIG. 9b), basically the same positive rate as when using CB7 was obtained in the knockdown cells (KD), and, moreover, the probe 5 positive/negative rate was approximately 1:1 when a sample of a 1:1 mixture of knockdown cells (KD) and non-knockdown cells (ctrl) was analyzed. Furthermore, virtually no dead cells were found in assay by SYTOX (registered trademark) Red Dead Cell Stain conducted simultaneously, and almost no cell death due to the effects of the probe, etc., was confirmed to occur. The top 25% (top) and bottom 25% (bottom) of fluorescent brightness were sorted out by FACS Aria II using a sample in which the positive and negative were well separated. The respective lysates were produced, Western blotting was carried out using the same, and the expression levels of ALDH3A1 were compared (FIG. 9c). When this was done, pronounced expression of ALDH3A1 was found in the probe 5 positive group and virtually no ALDH3A1 could be found in the negative group, in agreement with the results of flow cytometry.

The above showed that by using compound 5 in combination with an MRP inhibitor it is possible to detect cells having high ALDH3A1 activity. Cells having high/low ALDH3A1 activity can also be prepared respectively by using a cell sorter and then used in biological experiments.

1. A compound represented by general formula (I) or a salt thereof; 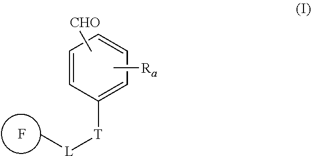
(where:
Rₐ is a hydrogen atom or a C1-4 alkyl group;
T is selected from the following divalent groups:
—N(R_{b})—,
—C(R_{b})—,
—O—
(R_{b} is a C1-4 alkyl group, R_{c} is a hydrogen atom or a C1-4 alkyl group, and each R_{c} may be the same or different);
L is a linker; 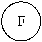
is a fluorophore, and
the fluorophore is selected from xanthene dyes, BODIPY fluorophores, or cyanine fluorophores) wherein said compound or salt thereof has a retention time on an HPLC chromatogram measured under the following conditions of longer than 6.9 minutes when said compound is in aldehyde form and of 6.9 minutes or less when said compound is in carboxylic acid form.
(HPLC conditions: taking solvent A to be 0.01 M ammonium formate/water and solvent B to be 80% acetonitrile 0.01 M ammonium formate/water, chromatography is carried out under conditions of 20% solvent B for 2.5 minutes followed by a 5-minute linear gradient of solvent B from 20% to 100% (flow rate 500 μL/min).)

2. The compound or salt thereof according to claim 1, wherein the linker is represented by Y—(S), Y is a linking group, and S, when present, is a crosslinking group.

3. The compound or salt thereof according to claim 2, wherein the linking group is selected from —CONH—, —R—CONH—, —COO—, —R—COO—, —RO—, or —R—CO— (where R is a hydrocarbon group).

4. The compound or salt thereof according to claim 2, wherein the crosslinking group is selected from C1-6 substituted or unsubstituted alkylene groups, —(CH₂—CH₂—O)ₘ— (m is 1 or 2), or a combination thereof.

5. The compound or salt thereof according to claim 1, wherein 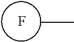
in formula (I) is represented by formula (II). 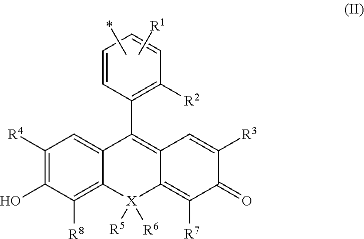
R¹ is a hydrogen atom or 1 to 4 of the same or different monovalent substituents present on the benzene ring;
R² is a hydrogen atom, monovalent substituent, or bond;
R³ and R⁴ are, each independently, a hydrogen atom, a C1-6 alkyl group, a halogen atom, or a bond;
R⁵ and R⁶ are, each independently, a C1-6 alkyl group, an aryl group, or a bond; provided that R⁵ and R⁶ are not present when X is an oxygen atom;
R⁷ and R⁸ are, each independently, a hydrogen atom, a C1-6 alkyl group, a halogen atom, or a bond;
X is an oxygen atom or silicon atom;
* represents a site of bonding with L of formula (I) at any position on the benzene ring,
where L bonds at at least one position selected from any position on the benzene ring, any of positions R²-R⁸.)

6. The compound or salt thereof according to claim 5 represented by formula (IIa). 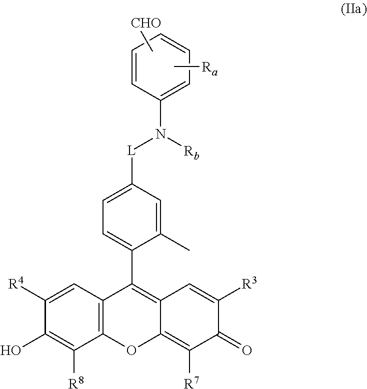
(In the formula, Rₐ, R_{b}, and L are as defined in formula (I), and R³, R⁴, R⁷, R⁸ are as defined in formula (II).)

7. The compound or salt thereof according to claim 1, wherein 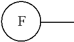
in formula (I) is represented by formula (III). 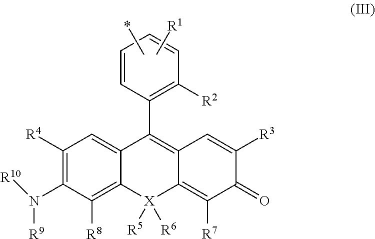
(in the formula, R¹-R⁸ and X are as defined in formula (II);
R⁹ and R¹⁰ are, each independently, a hydrogen atom, a C1-3 alkyl group, or a bond,
R⁹ and R¹⁰ together may form a 4- to 7-membered heterocyclyl including nitrogen atoms to which R⁹ and R¹⁰ are bonded,
R⁹ or R¹⁰ or both R⁹ and R¹⁰, together with R⁴ or R⁸, respectively, may form a 5- to 7-membered heterocyclyl or heteroaryl including a nitrogen atom to which R⁹ or R¹⁰ is bonded, may also contain 1-3 heteroatoms selected from the group consisting of an oxygen atom, nitrogen atom, or sulfur atom as ring members, and the heterocyclyl or heteroaryl may also be substituted by a C1-6 alkyl, C2-6 alkenyl, or C2-6 alkynyl, C6-10 aralkyl group, or C6-10 alkyl-substituted alkenyl group; and
* represents a site of bonding with L of formula (I) at any position on the benzene ring,
where L bonds at any position on the benzene ring, at at least one position selected from any of positions R2-R¹⁰).

8. The compound or salt thereof according to claim 1, wherein 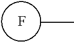
in formula (I) is represented by formula (IV). 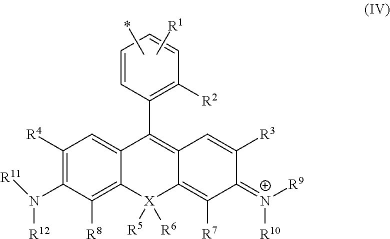
(in the formula, R¹-R⁸ and X are as defined in formula (II);
R⁹ and R¹⁰ are, each independently, a hydrogen atom or a C1-6 alkyl group,
R⁹ and R¹⁰ together may form a 4- to 7-membered heterocyclyl including nitrogen atoms to which R⁹ and R¹⁰ are bonded,
R⁹ or R¹⁰ or both R⁹ and R¹⁰, together with R³ or R⁷, respectively, may form a 5- to 7-membered heterocyclyl or heteroaryl including a nitrogen atom to which R⁹ or R¹⁰ is bonded, may also contain 1-3 heteroatoms selected from the group consisting of an oxygen atom, nitrogen atom, and sulfur atom as ring members, and the heterocyclyl or heteroaryl may also be substituted by a C1-6 alkyl, C2-6 alkenyl, or C2-6 alkynyl, C6-10 aralkyl group, or C6-10 alkyl-substituted alkenyl group;
R¹¹ and R¹² are, each independently, a hydrogen atom, a C1-3 alkyl group, or a bond,
R¹¹ and R¹² together may form a 4- to 7-membered heterocyclyl including nitrogen atoms to which R¹¹ and R¹² are bonded,
R¹¹ or R¹² or both R¹¹ and R¹², together with R⁴ or R⁸, respectively, may form a 5- to 7-membered heterocyclyl or heteroaryl including a nitrogen atom to which R¹¹ or R¹² is bonded, may also contain 1-3 heteroatoms selected from the group consisting of an oxygen atom, nitrogen atom, and sulfur atom as ring members, and the heterocyclyl or heteroaryl may also be substituted by a C1-6 alkyl, C2-6 alkenyl, or C2-6 alkynyl, C6-10 aralkyl group, or C6-10 alkyl-substituted alkenyl group; and
* represents a site of bonding with L of formula (I) at any position on the benzene ring,
where L bonds at at least one position selected from any position on the benzene ring, any of positions R²-R¹².)

9. The compound or salt thereof according to claim 1, wherein 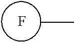
in formula (I) is represented by formula (V). 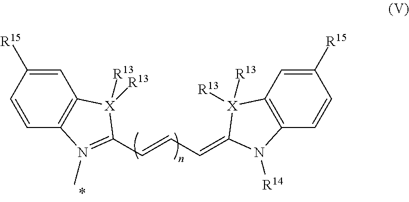
(in the formula,
R¹³ and R¹⁴ are, each independently, a hydrogen atom or a C1-6 alkyl group;
R¹⁵ is, each independently, a hydrogen atom, carboxyl group, or sulfonic acid group;
n is an integer of 1-3; and
* represents a site of bonding with L of formula (I).)

10. The compound or salt thereof according to claim 1, wherein 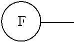
in formula (I) is represented by formula (VI). 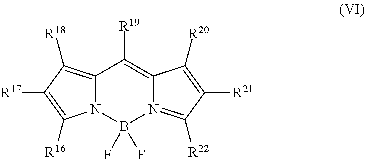
(in the formula, R¹⁶-R²² are, each independently, a hydrogen atom, a C1-6 alkyl group, a carbonyl group, an allyl group, an aryl group, a pyrrole group, a thiophene group, a furan group, a sulfonic acid group, a sulfonylamide group, a carboxyl group, a methoxy group, or a bond; and L bonds at at least one position selected from any of positions R¹⁶-R²².)

11. A fluorescent probe for ALDH3A1 detection including the compound or salt thereof according to claim 1.
